SEQUENCE LISTING (24 sequences)

SEQ ID NO: 1 - protein, 190 aa, Cowpea chlorotic mottle virus
MSTVGTGKLTRAQRRAAARKNKRNTRVVQPVIVEPIASGQGKAIKAWTGYSVSKWTASCAAAEAKVTSAITISLPNELSSERNKQLKVGRVLLWLGLLPSVSGTVKSCVTETQTTAAASFQVALAVADNSKDVVAAMYPEAFKGITLEQLTADLTIYLYSSAALTEGDVIVHLEVEHVRPTFDDSFTPVY

SEQ ID NO: 2 - DNA, 573 bp, Cowpea chlorotic mottle virus
atgtctacagtcggaacagggaagttaactcgtgcacaacgaagggctgcggcccgtaagaacaagcggaacactcgtgtggtccaacctgttattgtagaacccatcgcttcaggccaaggcaaggctattaaagcatggaccggttacagcgtatcgaagtggaccgcctcttgcgcggccgccgaagctaaagtaacctcggctataactatctctctccctaatgagctatcgtccgaaaggaacaagcagctcaaggtaggtagagttttattatggcttgggttgcttcccagtgttagtggcacagtgaaatcctgtgttacagagacgcagactactgctgctgcctcctttcaggtggcattagctgtggccgacaactcgaaagatgttgtcgctgctatgtaccccgaggcgtttaagggtataacccttgaacaactcaccgcggatttaacgatctacttgtacagcagtgcggctctcactgagggcgacgtcatcgtgcatttggaggttgagcatgtcagacctacgtttgacgactctttcactccggtgtattag

SEQ ID NO: 3 - DNA, 75 bp, Bacillus anthracis
agtaattctcgtaagaaacgttctacctctgctggccctaccgtgcctgatcgtgataatgatggcattcctgat

SEQ ID NO: 4 - protein, 25 aa, Bacillus anthracis
SNSRKKRSTSAGPTVPDRDNDGIPD

SEQ ID NO: 5 - DNA, 75 bp, Bacillus anthracis
agtcctgaagctcgtcatcctctcgtggctgcgtatcctattgtgcatgttgatatggaaaatattatcctctct

SEQ ID NO: 6 - protein, 25 aa, Bacillus anthracis
SPEARHPLVAAYPIVHVDMENIILS

SEQ ID NO: 7 - DNA, 75 bp, Bacillus anthracis
cgtattattttcaatggcaaagatctcaatctcgtggaacgtcgtattgctgctgtgaatccttctgatcctctc

SEQ ID NO: 8 - protein, 25 aa, Bacillus anthracis
RIIFNGKDLNLVERRIAAVNPSDPL

SEQ ID NO: 9 - DNA, 75 bp, Bacillus anthracis
cgtcaagatggcaaaaccttcattgatttcaaaaagtataatgataaactccctctctatatttctaatcctaat

SEQ ID NO: 10 - protein, 25 aa, Bacillus anthracis
RQDGKTFIDFKKYNDKLPLYISNPN

SEQ ID NO: 11 - protein, 25 aa, Eastern equine encephalomyelitis virus
DLDTHFTQYKLARPYIADCPNCGHS

SEQ ID NO: 12 - DNA, 75 bp, Eastern equine encephalomyelitis virus
gacctggacacccacttcacccagtacaagctggcccgcccgtacatcgccgactgcccgaactgcggccacagc

SEQ ID NO: 13 - protein, 25 aa, Eastern equine encephalomyelitis virus
GRLPRGEGDTFKGKLHVPFVPVKAK

SEQ ID NO: 14 - DNA, 75 bp, Eastern equine encephalomyelitis virus
ggccgcctgccgcgcggcgaaggcgacaccttcaagggcaagctgcacgtgccgttcgtgccggtgaaggccaag

SEQ ID NO: 15 - DNA, 56 bp, Eastern equine encephalomyelitis virus
cggggatcctggacctggacacccacttcacccagtacaagctggcccgcccgtac

SEQ ID NO: 16 - DNA, 57 bp, Eastern equine encephalomyelitis virus
cgcaggatcccgctgtggccgcagttcgggcagtcggcgatgtacgggcgggccagc

SEQ ID NO: 17 - DNA, 54 bp, Eastern equine encephalomyelitis virus
cggggatcctgggccgcctgccgcgcggcgaaggcgacaccttcaagggcaagc

SEQ ID NO: 18 - DNA, 54 bp, Eastern equine encephalomyelitis virus
cgcaggatccccttggccttcaccggcacgaacggcacgtgcagcttgcccttg

SEQ ID NO: 19 - DNA, 6 bp, artificial sequence
  palindromic hexamer of CpG sequence, can be chemically synthesized for immunostimmulatory sequence
  r (1)..(1): purine (preference for G)
  d (2)..(2): purine or T
  y (5)..(5): pyrimidine
  y (6)..(6): pyrimidine
rdcgyy

SEQ ID NO: 20 - DNA, 6 bp, artificial sequence
  CpG sequence can be chemically synthesized for immunostimmulatory sequence
gacgtc

SEQ ID NO: 21 - DNA, 6 bp, artificial sequence
  CpG sequence can be chemically synthesized for immunostimmulatory sequence
agcgct

SEQ ID NO: 22 - DNA, 6 bp, artificial sequence
  CpG sequence can be chemically synthesized for immunostimmulatory sequence
aacgtt

SEQ ID NO: 23 - DNA, 20 bp, artificial sequence
  CpG sequence can be chemically synthesized for immunostimmulatory sequence
tccatgacgttcctgacgtt

SEQ ID NO: 24 - DNA, 8 bp, artificial sequence
  CpG sequence can be chemically synthesized for immunostimmulatory sequence
aacgttcg

(19) United States
(12) Patent Application Publication
(10) Pub. No.: US 2007/0041999 A1
(43) Pub. Date: Feb. 22, 2007

(54) Production of multivalent virus like particles

(76) Inventors: Lada Rasochova, Del Mar, CA (US); Philip P. Phuoc Dao, San Diego, CA (US); Jamie P. Phelps, San Diego, CA (US)

Correspondence Address: KING & SPALDING LLP, 1180 PEACHTREE STREET, ATLANTA, GA, 30309-3521, US

(21) Appl. No.: 11/444,827

(22) Filed: Jun. 1, 2006

Related U.S. Application Data
(60) Provisional application No. 60/686,541, filed on Jun. 1, 2005.

(51) Int. Cl. A61K 39/12; C12N 7/01

(52) U.S. Cl. 424/204.1; 435/235.1; 977/802

(57) ABSTRACT

The present invention is directed to the production and in vitro assembly of recombinant viral capsid proteins into virus like particles. In particular, the present invention provides rapid, scalable, and cost efficient methods for the production of multivalent virus like particles utilizing separate populations of capsid fusion peptides containing differing antigenic peptide inserts that are combined in vitro to produce homogenous populations of multivalent virus like particles. The virus like particles produced according to the present invention can be utilized to induce an immunological response in human or animal.

### CROSS-REFERENCE TO RELATED APPLICATION

This application claims priority to U.S. Provisional application No. 60/686,541, filed Jun. 1, 2005.

### STATEMENT OF GOVERNMENT INTEREST

This application is under a United States Government contract with the National Institutes of Health, National Institute of Allergy and Infectious Disease (NIAID), Cooperative Agreement No. 1-U01-AI054641-01.

### FIELD OF THE INVENTION

The present invention is directed to the production and in vitro assembly of recombinant viral capsid proteins into virus like particles. In particular, the present invention provides rapid, scalable, and cost efficient methods for the production of multivalent virus like particles utilizing separate populations of capsid fusion peptides containing differing antigenic peptide inserts that are combined in vitro to produce multivalent virus like particles. The virus like particles produced according to the present invention can be utilized to induce an immunological response in humans or animals.

### BACKGROUND OF THE INVENTION

Vaccinations are one of the most effective and efficient ways to protect animals and humans from infections by pathogenic agents. Recently, the use of vaccine inoculations containing epitopes to more than one pathogenic agent have been examined. The rationale behind such a strategy includes the reduction in the number of inoculations required to induce immunity, which may reduce doctor's visits and increase compliance with recommended vaccine protocols. For example, the first pentavalent vaccine in the United States against Diphtheria, Tetanus, Pertussis, Polio, and Hepatitis B has been developed by GlaxoSmithKline under the commercial name Pediarix. Other multivalent vaccines in use or development include: Comvax, manufactured by Merck, which combines the hepatitis B and Hib vaccine into one shot; TriHIBit, manufactured by Aventis, which combines Hib and DTaP; and Twinrix, manufactured by GlaxoSmithKline, which combines hepatitis A and hepatitis B into one shot and is given as a three dose series. Other combination vaccines in development include single inoculants that combine: MMR and Varivax; DTaP and IPV; DTaP and hepatitis B; DTaP, IPV, and Hib (Pentavac); DTaP, hepatitis B, and Hib; DTaP, IPV, Hib and hepatitis B (Hexavac); and DTaP, Hib, IPV, hepatitis A, and hepatitis B.

Virus Like Particles

Virus like particles (VLPs) have been investigated as vaccine agents. In general, encapsidated viruses include a protein coat or “capsid” that is assembled to contain the viral nucleic acid. Many viruses have capsids that can be “self-assembled” from the individually expressed capsid proteins to form VLPs, both within the cell the capsid is expressed in (“in vivo assembly”) and outside of the cell after isolation and purification (“in vitro assembly”).

Virus like particles mimic the overall structure of a virus particle without the requirement of containing infectious material. VLPs can lack a viral DNA or RNA genome, but retain the three-dimensional structure of an authentic virus. VLPs have the ability to stimulate B-cell mediated responses, CD4 proliferative responses and cytotoxic T lymphocytes responses. See, Schirmbeck et al (1996) Virus like particles induce MHC class I-restricted T-cell responses. Lessons learned from the hepatitis B small surface antigen. Intervirology 39, 111-119; Paliard et al (2000) Priming of strong, broad, and long lived HIV type I p55gag-specific CD8+ cytotoxic T cells after administration of a virus like particle vaccine in rhesus macaques. AIDS Res. Hum. Retroviruses 16, 273-282; Murata et al. (2000) Immunization with hepatitis C virus like particles protects mice from recombinant hepatitis C virus-vaccinia infection. PNAS USA 100, 6753-6758.

VLPs have been produced for more than 30 different viruses that infect humans and other animals, including Norwalk, Hepatitis B and C, Papillomavirus, Parvovirus, and Influenza A, and a number of clinical trials in humans using VLPs are currently underway. See, Koutsky et al. (2002) “A controlled trial of a human papillomavirus type 16 vaccine,” NEJM 347:1645-1651; Pinto et al. (2003) “Cellular immune responses to human papillomavirus (HPV)-16 L1 in healthy volunteers immunized with recombinant HPV-16 L1 virus like particles,” J. Infect. Dis 188:327-338; Tacket et al. (2003) “Humoral, mucosal, and cellular immune responses to oral Norwalk virus like particles in volunteers,” Clin. Immunol. 108:241-247.

Virus like particles can also be manipulated to act as carrier molecules for the delivery of epitopes from other pathogenic agents. See, Noad et al. (2003) “Virus like particles as immunogens,” Trends in Microbiology 11(9), 438-444; Sadeyen et al. (2003) “Insertion of a foreign sequence on capsid surface loops of human papillomavirus type 16 virus like particles reduces their capacity to induce neutralizing antibodies and delineates a conformational neutralizing epitope,” Virology 309:32-40; WO 2005/005614; U.S. Patent Publication Nos. 2004/0033585 and 2005/0048082; U.S. Pat. Nos. 6,448,070; 6,110,466; 6,171,591; Brinkman et. al. (2004) “Recombinant murine polyoma virus-like-particles induce protective anti-tumour immunity,” Lett. Drug Des. & Disc. 1:137-147. A capsid protein can be modified to contain an antigenic peptide, generating a recombinant viral capsid protein-antigenic peptide fusion. This fusion capsid protein-antigenic peptide product can then be expressed in a host cell, assembled in vivo or in vitro to form recombinant viral or virus-like particles, and administered to a host in order to illicit an immune response.

Production of VLPs

The ideal multivalent VLP production method would allow for rapid, flexible, and controlled assembly of homogenous populations of VLPs containing multiple antigenic peptide inserts from different pathogenic agents, while being free of extemporaneous infectious viral nucleic acids. Current methods of constructing multivalent VLPs suffer from either: i) the lack of flexibility and control in the generation of the VLPs in vivo, which reduces the number of potential combinations of antigenic inserts due to inherent limitations in the capacity of capsid protein insertion, or ii) the production of non-homogeneous VLP populations due to the simple in vitro mixing of previously assembled populations of VLPs containing different inserts.

### SUMMARY OF THE INVENTION

The present invention provides for scalable in vitro virus like particle (VLP) assembly methods using recombinant viral capsid proteins containing antigenic peptide inserts and lacking full-length infectious viral nucleic acid genomes. The method includes assembling viral capsid proteins containing antigenic inserts into VLPs that lack full length infectious viral nucleic acid genomes. Specifically, the method includes mixing a first viral capsid protein containing at least one antigenic peptide insert in vitro with at least a second viral capsid protein containing at least one antigenic peptide insert, wherein at least one antigenic peptide insert of the second capsid fusion peptide is derived from a different antigenic peptide sequence, or a different pathogenic agent, than at least one antigenic peptide insert of the first capsid fusion peptide, and assembling the capsid proteins under proper conditions in vitro to form a virus like particle. The assembled virus like particle comprises at least two different antigenic sequence, providing a multivalent virus like particle. In some embodiments, the mixtures of the recombinant capsid fusion peptides containing different antigenic peptide inserts can be controlled so that specific ratios of desired antigenic peptides are achieved in the assembled virus like particles. In other embodiments, the VLP assembled according to the present invention does not contain full length infectious viral nucleic acids. In other embodiments, the VLP assembled according to the present invention does not contain viral nucleic acids. The current method allows for flexibility in producing virus like particles containing combinations of multiple antigenic peptides. These multivalent VLPs can be used in any number of applications, including vaccine strategies to illicit immunological responses in animals.

The current method utilizes mixtures of recombinant capsid fusion peptides containing antigenic peptide inserts to assemble a single population of multivalent VLPs that do not require infectious viral nucleic acids. Because the VLP is assembled in vitro with capsid proteins containing different antigenic peptides, the desired ratio of antigens contained in the VLP can be controlled. Using this technique, a wide array of combinations and ratios of capsid proteins containing different antigenic peptides can be mixed and quickly assembled to produce multivalent VLPs. Such a strategy allows for the rapid tailoring of a VLP's content to reflect a desirable antigenic makeup.

The current invention can utilize recombinant capsid fusion proteins derived from any source. For example, the recombinant capsid protein containing the insert can be derived from a previously assembled virus like particle. Such virus like particles, for example, may have been assembled in vivo, or in vitro. Alternatively, recombinant capsid proteins that have not been previously assembled into VLPs can be utilized. Furthermore, recombinant capsid proteins derived from previously assembled VLPs may be mixed with recombinant capsid proteins that have not been previously assembled into VLPs to produce multivalent VLPs.

The recombinant capsid proteins for use in the present invention can be generated in any host cell expression system that can produce such peptides, including, but not limited to, bacterial, yeast, insect, mammalian, and plant host cell systems, among others. In certain embodiments the capsid protein can be expressed in a prokaryotic host cell. In one embodiment, the prokaryotic host cell is a bacterial host cell. In some embodiments the capsid fusion peptides can be produced as soluble capsid proteins or insoluble inclusion bodies. In certain embodiments the recombinant capsid protein can be expressed in a Pseudomonas fluorescens cell as soluble capsid fusion proteins or in insoluble inclusion bodies. In another embodiment, the capsid protein is produced in a eukaryotic host cell. In a particular embodiment, the eukaryotic host cell is a plant cell. In another embodiment the capsid fusion peptides are produced in the whole plants.

The present invention provides for the mixing of different types of recombinant capsid fusion peptides, wherein the capsid fusion peptides selected for mixture contain at least one antigenic peptide that is not present in the other capsid fusion peptides to which it is being mixed. The antigenic peptide can be from the same or different pathogenic agents. In some embodiments, the capsid fusion peptides selected for mixture contain antigenic peptides from different pathogenic agents. In certain embodiments the mixture can contain at least two populations of recombinant capsid fusion peptides, wherein each recombinant capsid fusion peptide population contains at least one different antigenic peptide from a different pathogenic agent which is not present in any other recombinant capsid fusion peptide that it is mixed with. In another embodiment, the mixture contains more than two populations of recombinant capsid fusion peptides, wherein each recombinant capsid fusion peptide population contains at least one different antigenic peptide from at least one different pathogenic agent. In some embodiments, the capsid fusion peptides contain multiple antigenic peptide inserts. In some embodiments, the capsid fusion peptides contain antigenic peptides that target specific types of immune effector cells, including, but not limited to, epitopes directed to T cells, B cells, and CTL cells. In an additional embodiment, the mixture also contains a wild type capsid protein.

The viral capsid protein utilized in the present invention can be derived from any type of virus capable of re-assembling into a virus like particle. In certain embodiments all of the recombinant viral capsid proteins mixed and reassembled into VLPs can be derived from the same virus. In an alternative embodiment, the recombinant viral capsid proteins mixed and re-assembled into VLPs are derived from different viruses. For example, recombinant viral capsid proteins derived from different viruses with similar morphological capsid structure (i.e. icosahedral, helical, etc.) can be mixed and re-assembled into VLPs.

The present invention allows efficient and flexible generation of multivalent vaccines by allowing the ratio and composition of the multivalent vaccine to be controlled. Because the present invention provides for the desired epitope containing capsid fusion peptides to be mixed after isolation and purification from independent production in separate host cells, tight regulation of desired combinations can be achieved. The present invention allows for the re-assembly of recombinant capsid proteins into any desirable antigenic component ratio by adjusting the amounts of each population of recombinant capsid fusion peptide added to the mixture.

Another aspect of the present invention includes the insertion of pre-determined functional amino acid sequences other than antigenic peptides into the viral capsid protein. These sequences can have functions other than eliciting an immune response. A nonlimiting example of such a sequences is a targeting amino acid sequence, such as a receptor binding site, and the method includes mixing the capsid protein containing the targeting amino acid sequences with at least one capsid protein containing an antigenic peptide insert. In some embodiments, such a targeting amino acid insert may direct assembled particles to specific cells or enable entry into the cell.

An additional aspect of the present invention provides for the inclusion of immunostimulatory nucleic acid sequences such as CpG sequences in the assembled VLP particle produced by the methods described herein.

The present invention can provide methods of increasing the efficiency and scalable production of multivalent vaccines utilizing virus like particles containing antigenic inserts from more than one pathogenic agent.

The present invention can also provide methods of producing multivalent VLPs containing antigenic inserts from more than one pathogenic agent wherein the ratio of antigen agents contained in the VLP can be easily controlled.

Additionally, the present invention can produce multivalent VLPs that are free of a full-length infection viral nucleic acid genome.

### BRIEF DESCRIPTION OF THE FIGURES

FIG. 1 is a western blot of insoluble protein fractions from Pseudomonas fluorescens MB214 host cells with CCMV capsid fusion peptides engineered to express PA1, PA2, PA3, and PA4 peptide inserts in the insoluble fraction. Cells were lysed at 0, 12 and 44 hours. The chimeric capsid fusion peptide is indicated by arrow.

FIG. 2 is a western blot of soluble protein fractions from Pseudomonas fluorescens MB214 host cells with CCMV capsid fusion peptides engineered to express PA1, PA2, PA3, and PA4 peptide inserts in the soluble fraction. Cells were lysed at 0, 12 and 44 hours.

FIG. 3 is pictures of sucrose density gradients showing separation of CCMV-PA3 VLPs with and without RNA.

FIG. 4 is an SDS-PAGE gel of CCMV-PA1 and CCMV-PA2 VLP bands with and without RNA isolated from a sucrose density gradient.

FIG. 5 is an electron micrograph of VLPs reassembled from CCMV-PA4 in the absence of RNA.

FIG. 6 is a diagram of expression and reassembly of multivalent VLPs containing multiple Protective Antigen (“PA”) epitopes.

FIG. 7 is a diagram illustrating packaging of CpGs into VLPs during assembly reaction.

FIG. 8 are HPLC analyses of stages of VLP production. 8A illustrates an SEC-HPLC analysis of plant produced CCMV virus particles. 8B demonstrates disassembly of plant produced CCMV virus particles into dimers. 8C shows the reassembly of isolated CCMV dimers into VLPs.

FIG. 9 is an SEC-HPLC analysis of purified disassembled CCMV dimer and reassembled CCMV VLP containing encapsulated CpG oligonucleotides.

FIG. 10 shows pictures of 1.2% agarose gel stained with EtBr (top) and protein stain (bottom) of CCMV VLPs containing encapsulated standard CpG oligonucleotides or CpGs with protected backbone.

### DETAILED DESCRIPTION OF THE INVENTION

I. Recombinant Viral Capsid Proteins

Viral Capsids

Embodiments of the present invention provide for the production of multivalent virus like particles comprised of the mixing of populations of recombinant capsid fusion peptides containing at least one antigenic peptide in vitro, wherein each population of recombinant capsid fusion peptides contains at least one antigenic peptide that is not contained in the capsid fusion peptides that it is mixed with, and reassembling the mixed recombinant capsid fusion peptides in pre-determined ratios in vitro to form multivalent virus like particles. In some embodiments the antigenic peptides contained in the resultant VLPs can be derived from different pathogenic agents.

The term “multivalent” as used herein indicates the presence of at least two differing antigenic peptide sequences in the reassembled virus like particle.

Morphology

The current invention is not dependent on the type of virus used to derive the capsid protein. Any viral capsid protein, following insertion of an antigenic peptide, that is capable of re-assembling into a virus like particle, or cage structure, can be utilized in the present invention. In embodiments of the present invention, the amino acid sequence of the capsid can be selected from the capsids of viruses classified as having any morphology, including: icosahedral (including icosahedral proper, isometric, quasi-isometric, and geminate or “twinned”), polyhedral (including spherical, ovoid, and lemon-shaped), bacilliform (including rhabdo- or bullet-shaped, and fusiform or cigar-shaped), and helical (including rod, cylindrical, and filamentous); any of which may be tailed and/or may contain surface projections, such as spikes or knobs.

In embodiments of the present invention the capsid amino acid sequence can be selected from the capsids of entities that are helical in shape. In other embodiments the capsid amino acid sequence can be selected from the capsids of entities that are icosahedral. In certain embodiments the capsid amino acid sequence can be selected from the capsids of entities that are icosahedral proper. In certain embodiments the capsid amino acid sequence can be selected from the capsids of icosahedral viruses. In some embodiments the capsid amino acid sequence can be selected from the capsids of icosahedral plant viruses. However, in other embodiments the viral capsid can be derived from an icosahedral virus not infectious to plants. For example, in one embodiment, the virus is a virus infectious to mammals.

Generally, viral capsids of icosahedral viruses are composed of numerous protein sub-units arranged in icosahedral (cubic) symmetry. Native icosahedral capsids can be built up, for example, with 3 subunits forming each triangular face of a capsid, resulting in 60 subunits forming a complete capsid. Representative of this small viral structure is e.g. bacteriophage ØX174. Many icosahedral virus capsids contain more than 60 subunits. Many capsids of icosahedral viruses contain an antiparallel, eight-stranded beta-barrel folding motif. The motif has a wedge-shaped block with four beta strands (designated BIDG) on one side and four (designated CHEF) on the other. There are also two conserved alpha-helices (designated A and B), one is between betaC and betaD, the other between betaE and betaF.

Viruses

Viral taxonomies recognize the following taxa of encapsidated-particle entities:

Group I Viruses, i.e. the dsDNA viruses;

Group II Viruses, i.e. the ssDNA viruses;

Group III Viruses, i.e. the dsRNA viruses;

Group IV Viruses, i.e. the ssRNA (+)-stranded viruses with no DNA stage;

Group V Viruses, i.e. the ssRNA (−)-stranded viruses;

Group VI Viruses, i.e. the RNA retroid viruses, which are ssRNA reverse transcribing viruses;

Group VII Viruses, i.e. the DNA retroid viruses, which are dsDNA reverse transcribing viruses;

Deltaviruses;

Viroids; and

Satellite phages and Satellite viruses, excluding Satellite nucleic acids and Prions.

Members of these taxa are well known to one of ordinary skill in the art and are reviewed in: H. V. Van Regenmortel et al. (eds.), Virus Taxonomy: Seventh Report of the International Committee on Taxonomy of Viruses (2000) (Academic Press/Elsevier, Burlington Mass., USA); the Virus Taxonomy web-page of the University of Leicester (UK) Microbiology & Immunology Department at http://wwwmicro.msb.le.ac.uk/3035/Virusgroups.html; and the on-line “Virus” and “Viroid” sections of the Taxonomy Browser of the National Center for Biotechnology Information (NCBI) of the National Library of Medicine of the National Institutes of Health of the US Department of Health & Human Services (Washington, D.C., USA) at http://www.ncbi.nlm.nih.gov/Taxonomy/tax.html.

The amino acid sequence of the capsid may be selected from the capsids of any members of any of these taxa. Amino acid sequences for capsids of the members of these taxa may be obtained from sources, including, but not limited to, e.g.: the on-line “Nucleotide” (Genbank), “Protein,” and “Structure” sections of the PubMed search facility offered by the NCBI at http://www.ncbi.nlm.nih.gov/entrez/query.fcgi.

Embodiments of the present invention include wherein the capsid amino acid sequence can be selected from taxa members that are specific for at least one of the following hosts: fungi including yeasts, plants, protists including algae, invertebrate animals, vertebrate animals, and humans. In additional embodiments the capsid amino acid sequence can be selected from members of any one of the following taxa: Group I, Group II, Group III, Group IV, Group V, Group VII, Viroids, and Satellite Viruses. In certain embodiments the capsid amino acid sequence can be selected from members of any one of these seven taxa that are specific for at least one of the six above-described host types. In certain embodiments the capsid amino acid sequence can be selected from members of any one of Group II, Group III, Group IV, Group VII, and Satellite Viruses; or from any one of Group II, Group IV, Group VII, and Satellite Viruses. In one embodiment, the viral capsid is selected from Group IV or Group VII. In some embodiments the viral capsid is selected from a virus of Group IV.

The viral capsid sequence can be derived from a virus not a native infectious agent to the cell in which the capsid fusion peptide is produced. Embodiments of the present invention include wherein the cell does not include viral proteins from the particular selected virus other than the desired icosahedral capsids. In some embodiments the viral capsid is derived from a virus with a tropism to a different family of organisms than the cell in which the capsid fusion peptide is produced. In another embodiment, the viral capsid is derived from a virus with a tropism to a different genus of organisms than the host cell in which the capsid peptide is produced. In another embodiment, the viral capsid is derived from a virus with a tropism to a different species of organisms than the host cell in which the capsid fusion peptide is produced.

Embodiments of the present invention include wherein the viral capsid is selected form an icosahedral virus. The icosahedral virus can be selected from a member of any of the Papillomaviridae, Totiviridae, Dicistroviridae, Hepadnaviridae, Togaviridiae, Polyomaviridiae, Nodaviridae, Tectiviridae, Leviviridae, Microviridae, Sipoviridae, Nodaviridae, Picornoviridae, Parvoviridae, Calciviridae, Tetraviridae, and Satellite viruses.

In certain embodiments of the present invention, the sequence can be selected from members of any one of the taxa that are specific for at least one plant host. In some embodiments the icosahedral plant virus species can be a plant-infectious virus species that is or is a member of any of the Bunyaviridae, Reoviridae, Rhabdoviridae, Luteoviridae, Nanoviridae, Partitiviridae, Sequiviridae, Tymoviridae, Ourmiavirus, Tobacco Necrosis Virus Satellite, Caulimoviridae, Geminiviridae, Comoviridae, Sobemovirus, Tombusviridae, or Bromoviridae taxa. In some embodiments the icosahedral plant virus species is a plant-infectious virus species that is or is a member of any of the Luteoviridae, Nanoviridae, Partitiviridae, Sequiviridae, Tymoviridae, Ourmiavirus, Tobacco Necrosis Virus Satellite, Caulimoviridae, Geminiviridae, Comoviridae, Sobemovirus, Tombusviridae, or Bromoviridae taxa. In some embodiments, the icosahedral plant virus species is a plant infectious virus species that is or is a member of any of the Caulimoviridae, Geminiviridae, Comoviridae, Sobemovirus, Tombusviridae, or Bromoviridae. In other embodiments, the icosahedral plant virus species can be a plant-infectious virus species that is or is a member of any of the Comoviridae, Sobemovirus, Tombusviridae, or Bromoviridae. In yet additional embodiments, the icosahedral plant virus species can be a plant-infectious virus species that is a member of the Comoviridae or Bromoviridae family. In certain embodiments the viral capsid is derived from a species of the Bromoviridae taxa. In certain other embodiments the capsid is derived from an Ilarvirus or an Alfamovirus. In certain embodiments the viral capsid is derived from a Cowpea Mosaic Virus or a Cowpea Chlorotic Mottle Virus. In other embodiments the capsid is derived from a Tobacco streak virus, Brome mosaic virus, or an Alfalfa mosaic virus (AMV).

II. Antigenic Peptide Inserts

Size

Embodiments of the present invention include wherein the antigenic peptides or proteins operably linked to a viral capsid sequence contain at least two amino acids. The antigenic peptides can be of sufficient size to generate an immunological response when administered in an effective amount to an animal. The peptides can be at least 2, 3, 4, 5, 6, 7, 8, 9, 10, 11, 12, 13, 14, 15, 16, 17, 18, 19, 20, 30, 45, 50, 60, 65, 75, 85, 95, 96, 99 or more amino acids long.

Embodiments of the present invention include wherein the recombinant capsid fusion peptide contains at least one antigenic peptide. In an alternative embodiment, the recombinant capsid fusion peptide contains more than one antigenic peptide. In some embodiments, the antigenic peptide is composed of at least two, at least 5, at least 10, at least 15 or at least 20 separate antigenic peptides. In still another embodiment, the antigenic peptide is inserted into the viral capsid protein so that it is exposed on at least one surface loop when the capsid protein is reassembled to form virus-like particles.

Antigenic Peptides

The antigenic peptides for use in the present invention can be any peptide sequence capable of generating an immunological response. For example, the antigenic peptide can be a peptide epitope, hapten, or a related peptide (e.g., antigenic viral peptide; virus related peptide, e.g., HIV-related peptide, hepatitis-related peptide; antibody idiotypic domain, cell surface peptide; antigenic human, animal, protist, plant, fungal, bacterial, and/or archaeal peptide; allergenic peptide and allergen desensitizing peptide). The antigenic peptide can be selected from those that are antigenic peptides of human or animal pathogenic agents, including infectious agents, parasites, cancer cells, and other pathogenic agents. Such pathogenic agents also include the virulence factors and pathogenesis factors, e.g., exotoxins, endotoxins, et al., of those agents. The pathogenic agents may exhibit any level of virulence, i.e. they may be, e.g., virulent, avirulent, pseudo-virulent, semi-virulent, and so forth. In some embodiments the antigenic peptide can contain an epitopic amino acid sequence from the pathogenic agent(s). In additional embodiments the epitopic amino acid sequence can include that of at least a portion of a surface peptide of at least one such agent.

More than one antigenic peptide can be selected for insertion in a single capsid protein, in which case the resulting virus-like particles can present multiple different antigenic peptides from multiple different capsid fusion peptides. In some embodiments of a multiple antigenic peptide format, the various antigenic peptides can all be selected from a plurality of epitopes from the same pathogenic agent. In other embodiments of a multi-antigenic-peptide format, the various antigenic peptides selected can all be selected from a plurality of closely related pathogenic agents, for example, different strains, subspecies, biovars, pathovars, serovars, or genovars of the same species or different species of the same genus. In an alternative embodiment, the antigenic peptides for insertion in the capsid fusion peptide can be from non-related pathogenic agents.

Embodiments of the present invention include wherein the pathogenic agent(s) can belong to at least one of the following groups: Bacteria and Mycoplasma agents including, but not limited to, pathogenic: Bacillus spp., e.g., Bacillus anthracis; Bartonella spp., e.g., B. quintana; Brucella spp.; Burkholderia spp., e.g., B. pseudomallei; Campylobacter spp.; Clostridium spp., e.g., C. tetani, C. botulinum; Coxiella spp., e.g., C. burnetii; Edwardsiella spp., e.g., E. tarda; Enterobacter spp., e.g., E. cloacae; Enterococcus spp., e.g., E. faecalis, E. faecium; Escherichia spp., e.g., E. coli; Francisella spp., e.g., F. tularensis; Haemophilus spp., e.g., H. influenzae; Klebsiella spp., e.g., K pneumoniae; Legionella spp.; Listeria spp., e.g., L. monocytogenes; Meningococci and Gonococci, e.g., Neisseria spp.; Moraxella spp.; Mycobacterium spp., e.g., M leprae, M tuberculosis; Pneumococci, e.g., Diplococcus pneumoniae; Pseudomonas spp., e.g., P. aeruginosa; Rickettsia spp., e.g., R. prowazekii, R. rickettsii, R. typhi; Salmonella spp., e.g., S. typhi; Staphylococcus spp., e.g., S. aureus; Streptococcus spp., including Group A Streptococci and hemolytic Streptococci, e.g., S. pneumoniae, S. pyogenes; Streptomyces spp.; Shigella spp.; Vibrio spp., e.g., V. cholerae; and Yersinia spp., e.g., Y. pestis, Y. enterocolitica. Fungus and Yeast agents including, but not limited to, pathogenic: Alternaria spp.; Aspergillus spp.; Blastomyces spp., e.g., B. dermatiditis; Candida spp., e.g., C. albicans; Cladosporium spp.; Coccidiodes spp., e.g., C. immitis; Cryptococcus spp., e.g., C. neoformans; Histoplasma spp., e.g., H. capsulatum; and Sporothrix spp., e.g., S. schenckii.

Embodiments of the present invention include wherein the pathogenic agent(s) can be from a protist agent including, but not limited to, pathogenic: Amoebae, including Acanthamoeba spp., Amoeba spp., Naegleria spp., Eniamoeba spp., e.g., E. histolytica; Cryptosporidium spp., e.g., C. parvum; Cyclospora spp.; Encephalitozoon spp., e.g., E. intestinalis; Enterocytozoon spp.; Giardia spp., e.g., G. lamblia; Isospora spp.; Microsporidium spp.; Plasmodium spp., e.g., P. falciparum, P. malariae, P. ovale, P. vivax; Toxoplasma spp., e.g., T. gondii; and Trypanosoma spp., e.g., T brucei.

Embodiments of the present invention include wherein the pathogenic agent(s) can be from a parasitic agent (e.g., helminthic parasites) including, but not limited to, pathogenic: Ascaris spp., e.g., A. lumbricoides; Dracunculus spp., e.g., D. medinensis; Onchocerca spp., e.g., O. volvulus; Schistosoma spp.; Trichinella spp., e.g., T spiralis; and Trichuris spp., e.g., T. trichiura.

In other embodiments the pathogenic agent(s) can be from a viral agent including, but not limited to, pathogenic: Adenoviruses; Arenaviruses, e.g., Lassa Fever viruses; Astroviruses; Bunyaviruses, e.g., Hantaviruses, Rift Valley Fever viruses; Coronaviruses, Deltaviruses; Cytomegaloviruses, Epstein-Barr viruses, Herpes viruses, Varicella viruses; Filoviruses, e.g., Ebola viruses, Marburg viruses; Flaviruses, e.g., Dengue viruses, West Nile Fever viruses, Yellow Fever viruses; Hepatitis viruses; Influenzaviruses; Lentiviruses, T-Cell Lymphotropic viruses, other leukemia viruses; Norwalk viruses; Papillomaviruses, other tumor viruses; Paramyxoviruses, e.g., Measles viruses, Mumps viruses, Parainfluenzaviruses, Pneumoviruses, Sendai viruses; Parvoviruses; Picornaviruses, e.g., Cardioviruses, Coxsackie viruses, Echoviruses, Poliomyelitis viruses, Rhinoviruses, Other Enteroviruses; Poxviruses, e.g., Variola viruses, Vaccinia viruses, Parapoxviruses; Reoviruses, e.g., Coltiviruses, Orbiviruses, Rotaviruses; Rhabdoviruses, e.g., Lyssaviruses, Vesicular Stomatitis viruses; and Togaviruses, e.g., Rubella viruses, Sindbis viruses, Western Encephalitis viruses.

Embodiments of the present invention include wherein the antigenic peptide is selected from the group consisting of a Canine parvovirus peptide, Bacillus anthracis protective antigen (PA) antigenic peptide, and an Eastern Equine Encephalitis virus antigenic peptide. In other embodiments the antigenic peptide is the canine parvovirus-derived peptide. In additional embodiments the antigenic peptide is the Bacillus anthracis protective antigen (PA) antigenic peptide with any one of the amino acid sequence of SEQ. ID. NOs: 4, 6, 8, or 10. In yet other embodiments the antigenic peptide is an Eastern equine Encephalitis virus antigenic peptide with the amino acid sequence of one of SEQ. ID. NOs: 11 or 13.

The coding sequence for the antigenic peptide or peptides of interest can be inserted into the coding sequence for a viral capsid or coat protein in a predetermined site. In some embodiments the peptide is inserted into the capsid coding sequence so as to be expressed as a loop during formation of a VLP.

Peptides may be inserted at more than one insertion site in a capsid. Thus, peptides may be inserted in more than one surface loop motif of a capsid; peptides may also be inserted at multiple sites within a given loop motif. The individual functional and/or structural peptide(s) of the insert(s), and/or the entire peptide insert(s), may be separated by cleavage sites, i.e. sites at which an agent that cleaves or hydrolyzes protein can act to separate the peptide(s) from the remainder of the capsid structure or assemblage.

Peptides may be inserted within external-facing loop(s) and/or within internal-facing loop(s), i.e. within loops of the capsid that face respectively away from or toward the center of the capsid. Any amino acid or peptide bond in a surface loop of a capsid can serve as an insertion for the peptide. Typically, the insertion site can be selected at about the center of the loop, i.e. at about the position located most distal from the center of the tertiary structure of the folded capsid peptide. The peptide coding sequence may be operably inserted within the position of the capsid coding sequence corresponding to this approximate center of the selected loop(s). This includes the retention of the reading frame for that portion of the peptide sequence of the capsid that is synthesized downstream from the peptide insertion site.

In other embodiments the peptide can be inserted at the amino terminus of the capsid. The peptide can be linked to the capsid through one or more linker sequences. In some other embodiments the peptide can be inserted at the carboxy terminus of the capsid. The peptide can also be linked to the carboxy terminus through one or more linkers, which can be cleavable by chemical or enzymatic hydrolysis. In additional embodiments peptide sequences are linked at both the amino and carboxy termini, or at one terminus and at at least one internal location, such as a location that is expressed on the surface of the capsid in its three dimensional conformation. For other embodiments of the present invention, at least one antigenic peptide is expressed within at least one internal loop, or in at least one external surface loop of the VLP.

More than one loop of the viral capsid can be modified. In some embodiments the antigenic peptide is exposed on at least two surface loops of the virus-like particle. In other embodiments at least two antigenic peptides are inserted into a capsid protein and exposed on at least two surface loops of the viral capsid, cage or virus-like particle. In another embodiment, at least three antigenic peptides are inserted into the capsid protein and exposed on at least three surface loops of the virus-like particle. The recombinant peptides in the surface loops can have the same amino acid sequence. In additional embodiments, the amino acid sequence of the recombinant peptides in the surface loops differ.

The nucleic acid sequence encoding the viral capsid or proteins can also be modified to alter the formation of VLPs (see e.g. Brumfield, et al. (2004) J. Gen. Virol. 85: 1049-1053). For example, three general classes of modification are most typically generated for modifying VLP assembly. These modifications are designed to alter the interior, exterior or the interface between adjacent subunits in the assembled protein cage. To accomplish this, mutagenic primers can be used to: (i) alter the interior surface charge of the viral nucleic acid binding region by replacing basic residues (e.g. K, R) in the N terminus with acidic glutamic acids (Douglas et al., 2002b); (ii) delete interior residues from the N terminus (in CCMV, usually residues 4-37); (iii) insert a cDNA encoding an 11 amino acid peptide cell-targeting sequence (Graf et al., 1987) into a surface exposed loop; and (iv) modify interactions between viral subunits by altering the metal binding sites (in CCMV, residues 81/148 mutant).

Embodiments of the present invention include wherein the antigenic peptide can be inserted into the capsid from a Cowpea Chlorotic Mottle Virus (CCMV). In some embodiments the peptide can be inserted at amino acid 129 of the CCMV virus. In another embodiment, the peptide sequence can be inserted at amino acids 60, 61, 62 or 63 of the CCMV virus. In still another embodiment, the peptide can be inserted at both amino acids 129 and amino acids 60-63 of the CCMV virus.

Embodiments of the present invention include wherein a tag sequence adjacent to the antigenic peptide of interest, or linked to a portion of the viral capsid protein, can also be included. In embodiments of the present invention this tag sequence can allow for purification of the recombinant capsid protein fusion peptide. The tag sequence can be an affinity tag, such as a hexa-histidine affinity tag. In another embodiment, the affinity tag can be a glutathione-S-transferase molecule. The tag can also be a fluorescent molecule, such as YFP or GFP, or analogs of such fluorescent proteins. The tag can also be a portion of an antibody molecule, or a known antigen or ligand for a known binding partner useful for purification.

III. Recombinant Capsid Protein Fusion Peptide Production

The present invention contemplates the use of synthetic or any type of biological expression system to produce the recombinant capsid fusion peptides for use in the subsequent assembly of multivalent VLPs. Current methods of capsid protein expression include insect cell expression systems, bacterial cell expression systems such as E. coli, B. subtilus, and P. fluorescens, plant and plant cell culture expression systems, yeast expression systems such as S. cervisiae and P. Pastoris, and mammalian expression systems.

Embodiments of the present invention include wherein recombinant capsid fusion peptides are produced in plant cells or whole plants. In certain embodiments the capsid fusion peptides can be produced as soluble proteins. In another embodiment, the capsid fusion peptides are assembled into infectious virus particles. In an alternative embodiment, the capsid fusion peptides are assembled as VLPs.

Embodiments of the present invention can include wherein the recombinant capsid protein fusion peptides are produced in a bacterial cell culture. In embodiments the recombinant capsid fusion peptides can aggregate as insoluble inclusion bodies within the host cell. In an alternative embodiment, the capsid protein fusion peptides are produced as soluble molecules within the host cell. In other embodiments the recombinant capsid fusion peptides are produced in a Pseudomonad host cell, including a Pseudomonas fluorescens cell.

The recombinant capsid protein fusion peptides for use in the present invention can be produced in biological expression systems utilizing well-known techniques in the art. For example, nucleic acid constructs encoding a fusion peptide of a viral capsid protein operably linked to at least one antigenic peptide can be introduced into a host cell and expressed. Transcriptional and translational regulatory elements, such as transcriptional enhancer sequences, translational enhancer sequences, promoters, ribosomal entry sites, including internal ribosomal entry sites, activators, translational start and stop signals, transcription terminators, cistronic regulators, polycistronic regulators, tag sequences, such as nucleotide sequence “tags” and “tag” peptide coding sequences, which facilitates identification, separation, purification, or isolation of the expressed recombinant capsid protein fusion peptide, including His-tag, Flag-tag, T7-tag, S-tag, HSV-tag, B-tag, Strep-tag, polyarginine, polycysteine, polyphenylalanine, polyaspartic acid, (Ala-Trp-Trp-Pro)n, thioredoxin, beta-galactosidase, chloramphenicol acetyltransferase, cyclomaltodextrin gluconotransferase, CTP:CMP-3-deoxy-D-manno-octulosonate cytidyltransferase, trpE or trpLE, avidin, streptavidin, T7 gene 10, T4 gp55, Staphylococcal protein A, streptococcal protein G, GST, DHFR, CBP, MBP, galactose binding domain, Calmodulin binding domain, KSI, c-myc, ompT, ompA, pelB, NusA, ubiquitin, hex-histidine, glutathione-S-transferase, GFP, YFP, or analogs of such fluorescent proteins, antibody molecules, hemosylin A, or a known antigen or ligand for a known binding partner useful for purification can be covalently attached to the described sequence so that by action of the host cell, the regulatory elements can direct the expression of the recombinant capsid protein fusion peptide.

In a fermentation process, once expression of the recombinant capsid fusion peptide is induced, it is ideal to have a high level of production in order to maximize the production efficiency of the capsid fusion peptides.

IV. Purification of Recombinant Capsid Protein Fusion Peptides

Once the recombinant capsid protein fusion peptide, virus-like particles or cage-like structures are produced, they can then be isolated and purified to substantial purity by standard techniques well known in the art.

The isolation and purification techniques can depend on the host cell utilized to produce the capsid protein fusion peptides. Such techniques can include, but are not limited to, PEG, ammonium sulfate or ethanol precipitation, acid extraction, anion or cation exchange chromatography, phosphocellulose chromatography, hydrophobic interaction chromatography, affinity chromatography, nickel chromatography, hydroxylapatite chromatography, reverse phase chromatography, lectin chromatography, preparative electrophoresis, detergent solubilization, selective precipitation with such substances as column chromatography, immunopurification methods, size exclusion chromatograph, immunopurification methods, centrifugation, ultracentrifugation, density gradient centrifugation (for example, on a sucrose or on a cesium chloride (CsCl) gradient), ultrafiltration through a size exclusion filter, and any other protein isolation methods known in the art. For example, capsid protein fusion peptide having established molecular adhesion properties can be reversibly fused to a ligand. With the appropriate ligand, the capsid protein fusion peptide can be selectively adsorbed to a purification column and then freed from the column in a relatively pure form. The capsid protein is then removed by enzymatic activity. In addition, the capsid protein fusion peptide can be purified using immunoaffinity columns or Ni-NTA columns.

General techniques are further described in, for example, R. Scopes, Peptide Purification: Principles and Practice, Springer-Verlag: N.Y. (1982); Deutscher, Guide to Peptide Purification, Academic Press (1990); U.S. Pat. No. 4,511,503; S. Roe, Peptide Purification Techniques: A Practical Approach (Practical Approach Series), Oxford Press (2001); D. Bollag, et al., Peptide Methods, Wiley-Lisa, Inc. (1996); AK Patra et al., Peptide Expr Purif, 18(2): p/182-92 (2000); and R. Mukhija, et al., Gene 165(2): p. 303-6 (1995). See also, for example, Ausubel, et al. (1987 and periodic supplements); Deutscher (1990) “Guide to Peptide Purification,” Methods in Enzymology vol. 182, and other volumes in this series; Coligan, et al. (1996 and periodic Supplements) Current Protocols in Peptide Science Wiley/Greene, NY; and manufacturer's literature on use of peptide purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See also, for example, Hochuli (1989) Chemische Industrie 12:69-70; Hochuli (1990) “Purification of Recombinant Peptides with Metal Chelate Absorbent” in Setlow (ed.) Genetic Engineering, Principle and Methods 12:87-98, Plenum Press, NY; and Crowe, et al. (1992) QIAexpress: The High Level Expression & Peptide Purification System QIAGEN, Inc., Chatsworth, Calif.

In some embodiments, the capsid protein fusion peptides expressed in host cells, especially bacterial host cells, may form insoluble aggregates (“inclusion bodies”). Several protocols are suitable for purification of peptides from inclusion bodies. For example, purification of inclusion bodies typically involves the extraction, separation and/or purification of inclusion bodies by disruption of the host cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension is typically lysed using 2-3 passages through a French Press. The cell suspension can also be homogenized using a Polytron (Brinknan Instruments) or sonicated on ice. Alternate methods of lysing bacteria are apparent to those of skill in the art (see, e.g., Sambrook et al., supra; Ausubel et al., supra).

If necessary, the inclusion bodies can be solubilized, and the lysed cell suspension typically can be centrifuged to remove unwanted insoluble matter. Capsid protein fusion peptides that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Although guanidine hydrochloride and similar agents are denaturants, this denaturation is not irreversible and renaturation may occur upon removal (by dialysis, for example) or dilution of the denaturant. Other suitable buffers are known to those skilled in the art.

Alternatively, it is possible to purify the recombinant capsid protein fusion peptides, virus like particles, or cage structures from the host periplasm. After lysis of the host cell, when the recombinant peptide is exported into the periplasm of the host cell, the periplasmic fraction of the bacteria can be isolated by cold osmotic shock in addition to other methods known to those skilled in the art. To isolate recombinant peptides from the periplasm, for example, the bacterial cells can be centrifuged to form a pellet. The pellet can be resuspended in a buffer containing 20% sucrose. To lyse the cells, the bacteria can be centrifuged and the pellet can be resuspended in ice-cold 5 mM MgSO₄ and kept in an ice bath for approximately 10 minutes. The cell suspension can be centrifuged and the supernatant decanted and saved. The recombinant peptides present in the supernatant can be separated from the host peptides by standard separation techniques well known to those of skill in the art.

An initial salt fractionation can separate many of the unwanted host cell peptides (or peptides derived from the cell culture media) from the recombinant capsid protein fusion peptides of interest. One such example can be ammonium sulfate. Ammonium sulfate precipitates peptides by effectively reducing the amount of water in the peptide mixture. Peptides then precipitate on the basis of their solubility. The more hydrophobic a peptide is, the more likely it is to precipitate at lower ammonium sulfate concentrations. A typical protocol includes adding saturated ammonium sulfate to a peptide solution so that the resultant ammonium sulfate concentration is between 20-30%. This concentration can precipitate the most hydrophobic of peptides. The precipitate is then discarded (unless the peptide of interest is hydrophobic) and ammonium sulfate is added to the supernatant to a concentration known to precipitate the capsid protein fusion peptide of interest. The precipitate is then solubilized in buffer and the excess salt removed if necessary, either through dialysis or diafiltration. Other methods that rely on solubility of peptides, such as cold ethanol precipitation, are well known to those of skill in the art and can be used to fractionate complex capsid protein fusion peptide mixtures.

The molecular weight of a recombinant capsid protein fusion peptide can be used to isolate it from peptides of greater and lesser size using ultrafiltration through membranes of different pore size (for example, Amicon or Millipore membranes). As a first step, the capsid protein fusion peptide mixture can be ultrafiltered through a membrane with a pore size that has a lower molecular weight cut-off than the molecular weight of the recombinant capsid fusion peptide of interest. The retentate of the ultrafiltration can then be ultrafiltered against a membrane with a molecular cut off greater than the molecular weight of the capsid protein fusion peptide of interest. The recombinant capsid protein fusion peptide can pass through the membrane into the filtrate. The filtrate can then be chromatographed as described below.

Recombinant capsid protein fusion peptides can also be separated from other peptides on the basis of its size, net surface charge, hydrophobicity, and affinity for ligands. In addition, antibodies raised against the capsid proteins can be conjugated to column matrices and the capsid proteins immunopurified. All of these methods are well known in the art. It can be apparent to one of skill that chromatographic techniques can be performed at any scale and using equipment from many different manufacturers (e.g., Pharmacia Biotech).

V. Disassembly of Assembled Virus Like Particles

In one aspect of the present invention, recombinant capsid protein fusion peptides that have been previously assembled into virus like particles can be utilized in the present invention, wherein the virus like particles are disassembled, and the recombinant capsid protein fusion peptides of the virus like particles are isolated and purified and subsequently utilized in the formation of multivalent virus like particles.

Disassembly processes are well known in the art. For example, dissociation buffers containing Tris, EGTA, DTT, and NaCl may be utilized to disassemble the previously assemble virus like particles. See, for example, Brady et al. (1977) “Dissociation of polyoma virus by the chelation of calcium ions found associated with purified virions,” J. Virology 23(3):717-724. In addition, prolonged exposure to high levels of sulfhydryl reducing agents such as β-mercaptoethanol, glutathione, dithiothreitol, dithioerythritol, cysteine, hydrogen sulfide, and mixtures thereof, can be utilized to disassemble previously assembled VLPs. See, for example, U.S. Pat. No. 6,146,945. Dis-assembly methods, such as those described in the Examples, can also be used in the present invention.

VI. Re-Assembly of VLPs

Different populations of recombinant capsid fusion peptides containing antigenic peptide inserts can be mixed in vitro and re-assembled to form virus like particles. In one aspect of the present invention, viral capsid proteins containing antigenic inserts are re-assembled into VLPs that lack full-length infectious viral nucleic acid genomes. Full-length infectious viral nucleic acid is a genomic nucleic acid of a virus that contains all nucleotide sequences that are required for viral replication in the cell. These elements include (i) coding regions, (ii) non-coding regions, and (iii) regulatory regions. The viral genomic nucleic acid can be RNA or DNA. The non-coding regions may be located at the 5′ and 3′ ends of the viral nucleic acids or they may be located between coding regions. The coding regions can be overlapping or non-overlapping and may be multifunctional. Both the non-coding and coding regions can have regulatory functions and contain regulatory elements such as sequences required for virus replication, translation, or encapsidation and particle formation.

In additional embodiments the VLP can be re-assembled in the presence of viral RNA. In another embodiment, the VLP is re-assembled in the absence of viral nucleic acids. In another embodiment, the VLP is re-assembled in the presence of non-viral nucleic acids. In another embodiment, the VLP is re-assembled in the presence of an immunostimulatory nucleic acid sequences, such as a CpG sequence.

In one aspect of the present invention, separate populations of recombinant capsid fusion peptide populations containing different antigenic peptide inserts are mixed in vitro and assembled into multivalent virus like particles. The separate populations each contain at least one antigenic peptide insert that is not present in any other recombinant capsid fusion peptide that it is mixed with.

In one embodiment the recombinant capsid fusions are mixed in the same ratios, for example a 1:1, 1:1:1 ratio. In another embodiment the recombinant capsid fusions are mixed in different ratios, for example 1:2, 1:3, 1:2:1, 2:1:3:1. Ratios can be determined by the number of different types of capsid fusion peptides containing antigenic inserts included in the mixture. In some embodiments the mixture of recombinant capsid fusion peptides contains at least a first viral capsid protein containing at least one antigenic peptide insert, and a second viral capsid protein containing at least one antigenic peptide insert, wherein at least one antigenic peptide insert of the second capsid fusion peptide is derived from a different antigenic peptide sequence, or a different pathogenic agent than at least one antigenic peptide insert of the first capsid fusion peptide. In some embodiments of the present invention at least two populations of recombinant capsid fusion peptides containing inserts from antigenic peptides are mixed, wherein each population contains at least one antigenic peptide insert that is not present in the capsid fusion peptide it is mixed with. The antigenic peptide insert can be from the same or different pathogenic agents. In some embodiments 3, 4, 5, 6, 7, 8, 9, 10, 11, 12, 15, 18, 20, or more than 20 populations of recombinant capsid fusion peptides are mixed, wherein each population contains at least one antigenic peptide insert that is not present in any other recombinant capsid fusion peptide population that it is mixed with. In other embodiments of the present invention, capsid proteins containing non-antigenic peptide inserts can also be included in the mixture. Such non-antigenic peptide inserts include, but are not limited to, targeting peptides, peptides that act as immune system modulators, such as cytokines and chemokines, and peptides that act as adjuvants derived from synthetic means.

In addition, non-viral nucleic acid sequences may be added to the mixture, such as immunostimulatory sequences, which may be useful in enhancing the immune response to the antigenic peptide inserts contained in the VLPs. Immunostimulatory nucleic acids, such as CpG sequences, are short oligonucleotides that mimic the innate immune response to microbial DNA. CpGs contain one or more cytosine-phosphate-guanine (CpG) dinucleotide-containing motifs with unmethylated cytosine residues. DNA-containing unmethylated CpG motifs common in bacterial but not in mammalian DNA have been shown to induce strong TH1-polarized immune responses both in vitro and in vivo. While not wishing to be limited to a single theory, the induction of TH1 responses is thought to be a result of the ability of immunostimulatory sequences containing CpG (CpG oligodeoxynucleotides) to induce activation and secretion of IL-12 and IL-18 by macrophages and dendritic cells. These cytokines then synergize to induce IFN-gamma production by natural killer and T cells. In addition, CpGs cause immature dendritic cells to mature to professional antigen-presenting cells able to activate antigen-reactive naive T cells. CpGs are also capable of directly driving B lymphocytes to proliferate and to trigger immunoglobulin production.

In one embodiment, the re-assembled VLP includes am unmethylated CpG sequences within a palindromic hexamer that follows the formula 5′-R¹R²CGY¹Y²-3′ (SEQ ID NO:19), where R¹ is a purine (preference for G), R² is a purine or T, and Y¹ and Y² are pyrimidines. In one embodiment, the VLP includes a CpG sequence selected from the group consisting of 5′-GACGTC-3′ (SEQ ID NO:20), 5′-AGCGCT-3′ (SEQ ID NO:21), and 5′-AACGTT-3′ (SEQ ID NO:22), or a combination thereof. In one embodiment, the VLP includes a CpG sequence comprising 5′ TCC ATG ACG TTC CTG ACG TT 3′ (SEQ ID NO:23). In another embodiment, the CpG oligonucleotide sequence is AACGTTCG (SEQ ID NO:24).

The resultant virus like particle, or cage structure, that is formed following the re-assembly of the mixed populations of recombinant capsid fusion peptides, as described above, contains at least the same number of different antigenic peptides as populations mixed. For example, if two populations of recombinant capsid fusion peptides are mixed, with each population containing at least one antigenic peptide that is not present in the other population, then the resultant virus like particle can contain at least two differing antigenic peptide inserts.

In another aspect of the present invention, separate populations of recombinant capsid fusion peptides containing at least one antigenic peptide insert are mixed in vitro to form VLPs, wherein each population of recombinant capsid fusion peptides mixed contains at least one capsid protein derived from a different virus than any other recombinant capsid fusion peptide it is mixed with. In other embodiments the mixture of recombinant capsid fusion peptides can contain at least a first viral capsid protein containing at least one antigenic peptide insert, and a second viral capsid protein containing at least one antigenic peptide insert, wherein the capsid protein of the first viral capsid protein is derived from a different virus than the capsid protein of the second viral capsid protein. In other embodiments the antigenic peptide insert can be the same or different peptide sequence. In yet additional embodiments the antigenic peptide insert can be from the same or different pathogenic agent. In some embodiments 3, 4, 5, 6, 7, 8, 9, 10, 11, 12, 15, 18, 20, or more than 20 populations of recombinant capsid fusion peptides are mixed, wherein each population contains a derived viral capsid protein that is not present in any other recombinant capsid fusion peptide population that it is mixed with. In additional embodiments of the present invention, capsid proteins containing non-antigenic peptide inserts can be included in the mixture, as discussed above. In addition, non-viral nucleic acid sequences may be added to the mixture, such as CpG sequences, which may be useful in enhancing the immune response to the antigenic peptide inserts contained in the VLPs.

Embodiments of the present invention include wherein capsid fusion proteins that do not contain peptide inserts (wild type capsid proteins) can also be added to the above described mixtures. The wild type viral capsid proteins can be derived from any virus, including the same or a different virus than the one used to derive the capsid fusion peptide containing the antigenic peptide insert.

The re-assembly of the capsid fusion peptides can produce virus like particles or cage structures. In some embodiments the VLP or cage structure is a multimeric assembly of the mixed capsids, including from three to about 1000 or more capsids. In other embodiments the VLP or cage structure includes at least 30, at least 50, at least 60, at least 90, at least 120 capsids, or at least 200 capsids. In another embodiment, each VLP or cage structure includes at least 150 capsids, at least 160, at least 170, or at least 180 capsids.

Embodiments of the present invention include wherein the VLP can be re-assembled as an icosahedral structure. In another embodiment, the VLP is re-assembled in the same geometry as the native virus that the capsid sequence is derived of. In a separate embodiment, however, the VLP does not have the identical geometry of the native virus. In some embodiments, for example, the structure is produced in a particle formed of multiple capsids but not forming a native-type VLP structure. For example, a cage structure of as few as 3 viral capsids can be formed. In separate embodiments, cage structures of about 6, 9, 12, 15, 18, 21, 24, 27, 30, 33, 36, 39, 42, 45, 48, 51, 54, 57, or 60 capsids can be formed.

In some aspects, the present invention provides for the ability to control and direct the ratio of antigenic peptide inserts contained in the re-assembled VLP at the mixing stage. The ratios of antigenic peptide inserts contained in the VLP are adjustable through the amounts added to the mixture prior to assembly. In this way, the present invention may allow for tighter regulation and control of the amount of some antigenic peptides contained in a re-assembled VLP than that which is attainable when the VLP is assembled in vivo. Such control, for example, may be useful in a vaccine strategy that utilizes a VLP containing one antigenic peptide that is being used as an inoculant for the first time in an animal, and a second antigenic peptide that is being used as a “booster” because the antigenic peptide has previously been used as an inoculant in an animal. In this case, for example, the “booster” antigenic peptide may be present in a lesser amount than the other antigenic peptide, and, using the current method, the recombinant capsid fusion peptide population containing the “booster” antigenic peptide may be added to the mixture in a lesser amount than the population containing the other antigenic peptide insert. In other embodiments at least one of the re-assembled VLP structures includes at least one capsid fusion peptide from each population added to the mixture. In additional embodiments the re-assembled VLP structures include roughly equal ratios of each capsid fusion peptide from each population added to the mixture. Alternatively, the ratios can be adjusted as desired, wherein disproportionate ratios of mixtures are achieved.

Virus like particle assembly requires correctly folded capsid proteins. However, additional factors significant for VLP formulation and stability may exist, including pH, ionic strength, di-sulfide bonds, divalent cation bonding, among others. See, for example, Brady et al, (1977) “Dissociation of polyoma virus by the chelation of calcium ions found associated with purified virions,” J. Virol. 23(3):717-724; Gajardo et al, (1997) “Two proline residues are essential in the calcium binding activity of rotavirus VP7 outer capsid protein,” J. Virol., 71:2211-2216; Walter et al, (1975) “Intermolecular disulfide bonds: an important structural feature of the polyoma virus capsid,” Cold Spring Har. Symp. Quant. Biol., 39:255-257 (1975); Christansen et al, (1977) “Characterization of components released by alkali disruption of simian virus 40,” J. Virol., 21:1079-1084; Salunke et al, (1986) “Self-assembly of purified polyomavirus capsid protein VP1,” Cell 46:895-904; Salunke et al, (1989) “Polymorphism in the assembly of polyomavirus capsid protein VP,” Biophys. J., 56:887-900; Garcea et al, (1983) “Host range transforming gene of polyoma virus plays a role in virus assembly,” Proc. Natl. Acad. Sci. USA, 80:3613-3617; Xi et al, (1991) “Baculovirus expression of the human papillomavirus type 16 capsid proteins: detection of L1-L2 protein complexes,” J. Gen. Virol., 72:2981-2988. Techniques that may be utilized for the re-assembly are well known in the art, and include, but are not limited to, techniques as described in the Examples.

The re-assembly of the capsid fusion peptides produces virus like particles or cage structures. In other embodiments the VLP or cage structure is a multimeric assembly of the mixed capsid fusion peptides, including from three to about 1000 or more capsids. In additional embodiments the VLP or cage structure includes at least 30, at least 50, at least 60, at least 90, at least 120 capsids, or at least 200 capsids. In another embodiment, each VLP or cage structure includes at least 150 capsids, at least 160, at least 170, or at least 180 capsids.

Embodiments of the present invention include wherein the VLP is re-assembled as an icosahedral structure. In other embodiments the VLP can be re-assembled in the same geometry as the native virus that the capsid sequence is derived of In additional embodiments the VLP does not have the identical geometry of the native virus. In some embodiments, for example, the structure is produced in a particle formed of multiple capsids but not forming a native-type VLP structure. For example, a cage structure of as few as 3 viral capsids can be formed. In separate embodiments, cage structures of about 6, 9, 12, 15, 18, 21, 24, 27, 30, 33, 36, 39, 42, 45, 48, 51, 54, 57, or 60 capsids can be formed.

VII. Vaccinations

In some aspects of the present invention, the re-assembled multivalent VLPs can be utilized in a strategy to induce an immune response in an animal or a human. Embodiments of the present invention include wherein the viral capsid protein utilized in the capsid fusion peptide is derived from a CCMV virus. In some embodiments the antigenic inserts contained in the VLPs include the “Protective Antigen” from Bacillus anthracis, or, alternatively, the E2 glycoprotein of the Eastern Equine Encephalitis.

In general, an effective quantity of VLP is administered to an animal or human which is sufficient for inducing an immune response. The amount administered to induce such a response can be determined by techniques generally known in the art. In some embodiments the quantity of VLP administered is advantageously between 10 and 500 μg of per individual animal or human. The quantity of the VLP administered may vary as a function of the administration route and of the weight of the individual.

The vaccine containing the VLP may be administered via a pharmaceutically acceptable carrier. The pharmaceutically acceptable carrier can be any physiological carrier known to those of ordinary skill in the art useful in formulating pharmaceutical. In some embodiments the pharmaceutical carrier can be a liquid and the vaccine containing the VLP would be in the form of a solution. In a further embodiment, the pharmaceutical carrier is a gel and the vaccine containing the VLP is in the form of a suppository or a cream. In yet a further embodiment, the vaccine containing the VLP may be formulated as a part of a pharmaceutically acceptable transdermal patch.

Liquid carriers are used in preparing solutions, suspensions, emulsions, syrups, elixirs and pressurized compositions. The VLP can be suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (partially containing additives as above, e.g. cellulose derivatives, sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate and isopropyl myristate. Sterile liquid carriers are useful in sterile liquid form compositions for parenteral administration. The liquid carrier for pressurized compositions can be halogenated hydrocarbon or other pharmaceutically acceptable propellent. Generally, the liquid carrier does not interfere with the folding of the reassembled VLP.

The vaccine containing the VLP can be administered using any technique currently utilized in the art, including, for example, orally, mucosally, intravenously, intramuscularly, intrathecally, epidurally, intraperitoneally or subcutaneously. In some embodiments the VLP is delivered mucosally through the nose or mouth. In other embodiments the reassembled VLP is comprised of a capsid protein derived from CCMV, and delivered mucosally.

### EXAMPLES

In these examples, the cowpea chlorotic mottle virus (CCMV) has been used as a peptide carrier and Pseudomonas fluorescens has been used as the expression host. CCMV is a member of the bromovirus group of the Bromoviridae. Bromoviruses are 25-28 nm diameter icosahedral viruses with a four-component, positive sense, single-stranded RNA genome. RNA1 and RNA2 code for replicase enzymes. RNA3 codes for a protein involved in viral movement within plant hosts. RNA4 (a subgenomic RNA derived from RNA 3), i.e. sgRNA4, codes for the 20 kDa capsid protein (CP), SEQ ID NO: 1.

Wild type CCMV coat protein encoded by sgRNA4
(SEQ ID NO:1)

Met Ser Thr Val Gly Thr Gly Lys Leu Thr Arg Ala
Gln Arg Arg Ala Ala Ala Arg Lys Asn Lys Arg Asn
Thr Arg Val Val Gln Pro Val Ile Val Glu Pro Ile
Ala Ser Gly Gln Gly Lys Ala Ile Lys Ala Trp Thr
Gly Tyr Ser Val Ser Lys Trp Thr Ala Ser Cys Ala
Ala Ala Glu Ala Lys Val Thr Ser Ala Ile Thr Ile
Ser Leu Pro Asn Glu Leu Ser Ser Glu Arg Asn Lys
Gln Leu Lys Val Gly Arg Val Leu Leu Trp Leu Gly
Leu Leu Pro Ser Val Ser Gly Thr Val Lys Ser Cys
Val Thr Glu Thr Gln Thr Thr Ala Ala Ala Ser Phe
Gln Val Ala Leu Ala Val Ala Asp Asn Ser Lys Asp
Val Val Ala Ala Met Tyr Pro Glu Ala Phe Lys Gly
Ile Thr Leu Glu Gln Leu Thr Ala Asp Leu Thr Ile
Tyr Leu Tyr Ser Ser Ala Ala Leu Thr Glu Gly Asp
Val Ile Val His Leu Glu Val Glu His Val Arg Pro
Thr Phe Asp Asp Ser Phe Thr Pro Val Tyr

Each CCMV particle contains up to about 180 copies of the CCMV CP. An exemplary DNA sequence encoding the CCMV CP is shown in SEQ ID NO. 2.

Exemplary DNA sequence encoding the CCMV CP
(SEQ ID NO:2)

atg tct aca gtc gga aca ggg aag tta act cgt gca
caa cga agg gct gcg gcc cgt aag aac aag cgg aac
act cgt gtg gtc caa cct gtt att gta gaa ccc atc
gct tca ggc caa ggc aag gct att aaa gca tgg acc
ggt tac agc gta tcg aag tgg acc gcc tct tgc gcg
gcc gcc gaa gct aaa gta acc tcg gct ata act atc
tct ctc cct aat gag cta tcg tcc gaa agg aac aag
cag ctc aag gta ggt aga gtt tta tta tgg ctt ggg
ttg ctt ccc agt gtt agt ggc aca gtg aaa tcc tgt
gtt aca gag acg cag act act gct gct gcc tcc ttt
cag gtg gca tta gct gtg gcc gac aac tcg aaa gat
gtt gtc gct gct atg tac ccc gag gcg ttt aag ggt
ata acc ctt gaa caa ctc acc gcg gat tta acg atc
tac ttg tac agc agt gcg gct ctc act gag ggc gac
gtc atc gtg cat ttg gag gtt gag cat gtc aga cct
acg ttt gac gac tct ttc act ccg gtg tat tag

The crystal structure of CCMV has been solved. This structure provides a clearer picture of the coat protein interactions that appear to be critical to particle stability and dynamics and has been helpful in guiding rational design of insertion sites. Previous studies have demonstrated that CCMV coat proteins can be genetically modified to carry heterologous peptides without interfering with their ability to form particles. A number of suitable insertion sites have been identified.

It is thought that a total of up to about 180 copies of a heterologous peptide unit (whether individual peptide or concatemer) can be inserted into the CCMV particle if a single insertion site in the CCMV CP is used. Insertion sites identified within CCMV CP to date can accommodate peptides of various lengths. In addition, multimeric forms of the peptides can be inserted into insertion sites. Furthermore, multiple insertion sites can be used at the same time to express the same or different peptides in/on the same particle. The peptide inserts can be about 200 amino acid residues or less in length, more typically up to or about 180, even more typically up to or about 150, still more typically up to or about 120, and yet more typically up to or about 100 amino acid residues in length. In some embodiments the peptide inserts can be about 5 or more amino acid residues in length. In other embodiments, the peptide inserts can be about 5 to about 200, about 5 to about 150, about 5 to about 120, more typically about 5 to about 100 amino acid residues in length.

Materials and Methods

Unless otherwise noted, standard techniques, vectors, control sequence elements, and other expression system elements known in the field of molecular biology are used for nucleic acid manipulation, transformation, and expression. Such standard techniques, vectors, and elements can be found, for example, in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); and Bukhari et al. (eds.), DNA Insertion Elements, Plasmids and Episomes (1977) (Cold Spring Harbor Laboratory Press, NY).

Plasmid Map Constructions

All plasmid maps were constructed using VECTORNTI (InforMax Inc., Frederick, Md., USA).

DNA Extractions

All plasmid DNA extractions from E. coli were performed using the mini, midi, and maxi kits from Qiagen (Germany) according to the manufacturer instructions.

Experimental Strategy

The following procedures were followed. P. fluorescens host cells were transformed with expression plasmids encoding chimeric viral coat protein-target peptide insert fusions. Transformed cells were grown to the desired density and induced to express the chimeric viral coat protein-peptide fusions. Cells were then lysed and their contents analyzed.

### Example 1

### Peptide Synthesis and Cloning into CCMV CP

1.A. Protective Antigen Cloning

Four different Bacillus anthracis protective antigen (“PA”) peptides (PA1-PA4) were independently expressed in CCMV VLPs. Nucleic acids encoding PA1-PA4 were synthesized by SOE (splicing-by-overlap-extension) of synthetic oligonucleotides. Each of the inserts was synthesized by over-lapping DNA oligos with the thermocycling program detailed below:

PCR PROTOCOL

| Reaction Mix (100 μL total volume) | | | | Thermocycling Steps | | | |
|---|---|---|---|---|---|---|---|
| 10 | μl | 10× PT HIFI buffer * | Step 1 | 1 | Cycle | 2 min. | 94° C. |
| 4 | μL | 50 mM MgSO₄ * | Step 2 | 35 | Cycles | 30 sec. | 94° C. |
| 2 | μL | 10 mM dNTPs * | Step 3 | 1 | Cycle | 30 sec. | 55° C. |
| 0.25 | ng | Each Primer | Step 4 | 1 | Cycle | 1 min. | 68° C. |
| 1-5 | ng | Template DNA | | | | 10 min. | 70° C. |
| 1 | μL | PT HIFI Taq DNA Polymerase * | | | | Maintain | 4° C. |

| | |
|---|---|
| Remainder | Distilled De-ionized H₂O (ddH₂O) |

* (from Invitrogen Corp, Carlsbad, CA, USA, hereinafter “Invitrogen”)

The resulting nucleic acids contained BamHI recognition site termini. The nucleotide sequences encoding, and the amino acid sequences of, these PA peptides were respectively as follows: 1) for PA1, SEQ ID NOs: 3 and 4; 2) for PA2, SEQ ID NOs: 5 and 6; 3) for PA3, SEQ ID NOs: 7 and 8; and) for PA4, SEQ ID NOs: 9 and 10. The resulting nucleic acids were digested with BamHI to create adhesive ends for cloning into shuttle vector. Each of the resulting PA inserts was cloned in the pESC-CCMV129BamHI shuttle plasmid at the BamHI site of the CCMV129 CDS. Each resulting shuttle plasmid was digested with SpeI and XhoI restriction enzymes. Each of the desired chimeric CCMV129-PA-encoding fragments was isolated by gel purification.

| | |
|---|---|
| PA1 | |
| Nucleic Acid | 5′-agt aat tct cgt aag aaa cgt tct |
| Sequence | acc tct gct ggc cct acc gtg cct gat |
| (SEQ ID NO:3) | cgt gat aat gat ggc att cct gat-3′ |
| Amino Acid | Ser Asn Ser Arg Lys Lys Arg Ser Thr |
| Sequence | Ser Ala Gly Pro Thr Val Pro Asp Arg |
| (SEQ ID NO:4) | Asp Asn Asp Gly Ile Pro Asp |
| PA2 | |
| Nucleic Acid | 5′-agt cct gaa gct cgt cat cct ctc |
| Sequence | gtg gct gcg tat cct att gtg cat gtt |
| (SEQ ID NO:5) | gat atg gaa aat att atc ctc tct-3′ |
| Amino Acid | Ser Pro Glu Ala Arg His Pro Leu Val |
| Sequence | Ala Ala Tyr Pro Ile Val His Val Asp |
| (SEQ ID NO:6) | Met Glu Asn Ile Ile Leu Ser |
| PA3 | |
| Nucleic Acid | 5′-cgt att att ttc aat ggc aaa gat |
| Sequence | ctc aat ctc gtg gaa cgt cgt att gct |
| (SEQ ID NO:7) | gct gtg aat cct tct gat cct ctc-3′ |
| Amino Acid | Arg Ile Ile Phe Asn Gly Lys Asp Leu |
| Sequence | Asn Leu Val Glu Arg Arg Ile Ala Ala |
| (SEQ ID NO:8) | Val Asn Pro Ser Asp Pro Leu |
| PA4 | |
| Nucleic Acid | 5′-cgt caa gat ggc aaa acc ttc att |
| Sequence | gat ttc aaa aag tat aat gat aaa ctc |
| (SEQ ID NO:9) | cct ctc tat att tct aat cct aat-3′ |
| Amino Acid | Arg Gln Asp Gly Lys Thr Phe Ile Asp |
| Sequence | Phe Lys Lys Tyr Asn Asp Lys Leu Pro |
| (SEQ ID NO:10) | Leu Tyr Ile Ser Asn Pro Asn |

The resulting chimeric CCMV129-PA polynucleotides were each then inserted into the pMYC1803 expression plasmid in place of the buibui coding sequence, in operable attachment to the tac promoter. The resulting expression plasmid was screened by restriction digest with SpeI and XhoI for presence of the insert.

1.B. E2 Glycoprotein of the Eastern Equine Encephalitis

Two different EEE peptides (EEE-1-25 and EEE-238-262) were independently expressed in CCMV VLPs, representing 25 AA peptides of the E2 glycoprotein of the Eastern Equine Encephalitis Virus.

| | |
|---|---|
| EEE-1-25 peptide sequence: | |
| DLDTHFTQYKLARPYIADCPNCGHS | (SEQ. ID. NO:11) |
| EEE-1-25 nucleic acid sequence: | |
| 5′-gacctggacacccacttcacccagtacaagc | (SEQ. ID. NO:12) |
| tggcccgcccgtacatcgccgactgcccgaactg | |
| cggccacagc-3′ | |
| EEE-238-262 peptide sequence: | |
| GRLPRGEGDTFKGKLHVPFVPVKAK | (SEQ. ID. NO:13) |
| EEE-238-262 nucleic acid sequence: | |
| 5′ ggccgcctgccgcgcggcgaaggcgacacct | (SEQ ID NO:14) |
| tcaagggcaagctgcacgtgccgttcgtgccggt | |
| gaaggccaag-3′ | |

Nucleic acids encoding EEE-1-25 and EEE-238-262 were synthesized by SOE of synthetic oligonucleotides. The resulting nucleic acids contained BamHI recognition site termini. The sense and anti-sense oligonucleotide primers for synthesis of the inserts included the BamHI restriction sites and were as follows:

| | |
|---|---|
| EEE1.S: | |
| 5′-cgg gga tcc tgg acc tgg aca ccc | (SEQ ID NO:15) |
| act tca ccc agt aca agc tgg ccc gcc | |
| cgt ac-3′ | |
| EEE1.AS: | |
| 5′-cgc agg atc ccg ctg tgg ccg cag | (SEQ ID NO:16) |
| ttc ggg cag tcg gcg atg tac ggg cgg | |
| gcc agc-3′ | |
| EEE2.S: | |
| 5′-cgg gga tcc tgg gcc gcc tgc cgc | (SEQ ID NO:17) |
| gcg gcg aag gcg aca cct tca agg gca | |
| agc-3′ | |
| EEE2.AS: | |
| 5′-cgc agg atc ccc ttg gcc ttc acc | (SEQ ID NO:18) |
| ggc acg aac ggc acg tgc agc ttg ccc | |
| ttg-3′ | |

The resulting nucleic acids were digested with BamHI to create adhesive ends for cloning into the pESC-CCMV129BamHI shuttle plasmid.

Each of the resulting EEE inserts was cloned in the pESC-CCMV129BamHI shuttle plasmid at the BamHI site of the CCMV129 CDS. Each resulting shuttle plasmid was digested with SpeI and XhoI restriction enzymes. Each of the desired chimeric CCMV-129-EEE-encoding fragments was isolated by gel purification.

The resulting chimeric CCMV129-EEE polynucleotide fragments were each then inserted into the pMYC 1803 expression plasmid restricted with SpeI and XhoI in place of the buibui coding sequence, in operable attachment to the tac promoter. The resulting expression plasmid was screened by restriction digest with SpeI and XhoI for presence of the insert.

### Example 2

### Expression of Recombinant CCMV Capsid Fusion Peptides

The CCMV129 fusion peptide expression plasmids were transformed into Pseudomonas fluorescens MB214 host cells according to the following protocol. Host cells were thawed gradually in vials maintained on ice. For each transformation, 1 μL purified expression plasmid DNA was added to the host cells and the resulting mixture was swirled gently with a pipette tip to mix, and then incubated on ice for 30 min. The mixture was transferred to electroporation disposable cuvettes (BioRad Gene Pulser Cuvette, 0.2 cm electrode gap, cat no. 165-2086). The cuvettes were placed into a Biorad Gene Pulser pre-set at 200 Ohms, 25 μfarads, 2.25 kV. Cells were pulse cells briefly (about 1-2 sec). Cold LB medium was then immediately added and the resulting suspension was incubated at 30° C. for 2 hours. Cells were then plated on LB tet15 (tetracycline-supplemented LB medium) agar and grown at 30° C. overnight.

One colony was picked from each plate and the picked sample was inoculated into 50 mL LB seed culture in a baffled shake flask. Liquid suspension cultures were grown overnight at 30° C. with 250 rpm shaking. 10 mL of each resulting seed culture was then used to inoculate 200 mL of shake-flask medium (i.e. yeast extracts and salt with trace elements, sodium citrate, and glycerol, pH 6.8) in a 1 liter baffled shake flask. Tetracycline was added for selection. Inoculated cultures were grown overnight at 30° C. with 250 rpm shaking and induced with IPTG for expression of the CCMV129-fusion peptide chimeric coat proteins. 1 mL aliquots from each shake-flask culture were then centrifuged to pellet the cells. Cell pellets were resuspended in 0.75 mL cold 50 mM Tris-HCl, pH 8.2, containing 2 mM EDTA. 0.1% volume of 10% TritonX-100 detergent was then added, followed by an addition of lysozyme to 0.2 mg/mL final concentration. Cells were then incubated on ice for 2 hours, at which time a clear and viscous cell lysate should be apparent.

To the lysates, 1/200 volume 1M MgCl₂ was added, followed by an addition of 1/200 volume 2 mg/mL DNAseI, and then incubation on ice for 1 hour, by which time the lysate should have become a much less viscous liquid. Treated lysates were then spun for 30 min at 4° C. at maximum speed in a tabletop centrifuge and the supernatants were decanted into clean tubes. The decanted supernatants are the “soluble” protein fractions. The remaining pellets were then resuspended in 0.75 mL TE buffer (10 mM Tris-Cl, pH 7.5, 1 mM EDTA). The resuspended pellets are the “insoluble” fractions.

These “soluble” and “insoluble” fractions were then electrophoresed on NuPAGE 4-12% Bis-Tris gels (from Invitrogen, Cat. NP0323), having 1.0 mm×15 wells, according to manufacturer's specification. 5 ul of each fraction were combined with 5 ul of 2× reducing SDS-PAGE loading buffer, and boiled for 5 minutes prior to running on the gel. The gels were stained with SimplyBlue Safe Stain, (from Invitrogen, Cat. LC6060) and destained overnight with water. Western blot detection employed CCMV IgG (Accession No. AS0011 from DSMZ, Germany) and the WESTERN BREEZE kit (from Invitrogen, Cat. WB7105), following manufacturer's protocols.

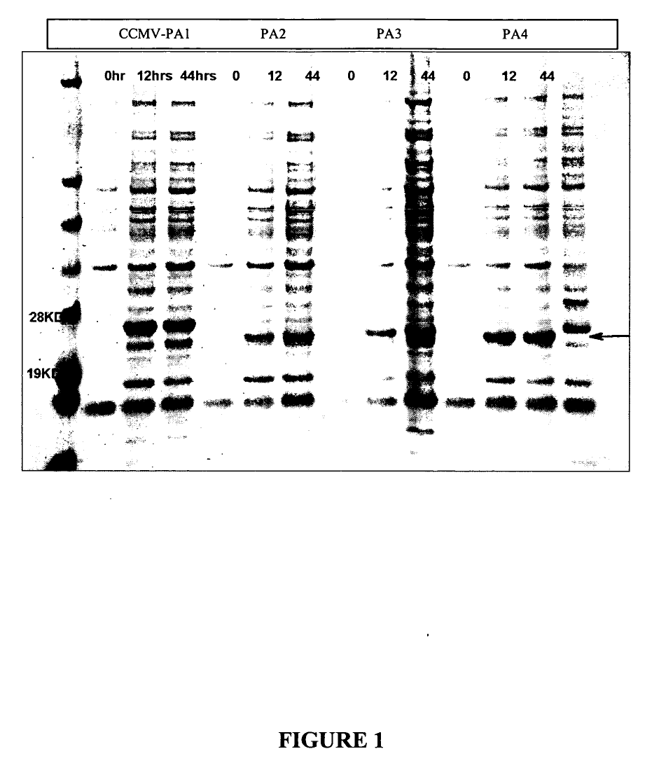

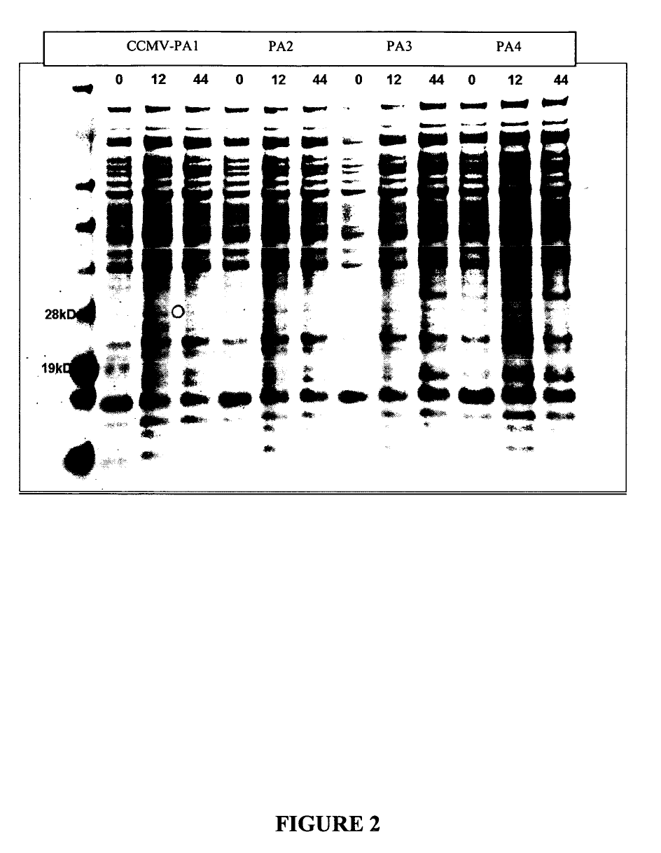

FIG. 1 shows the expression of recombinant CCMV capsid proteins engineered to express PA1, PA2, PA3, and PA4 peptide inserts in the insoluble fraction. The recombinant capsid fusion peptide is indicated by arrow. FIG. 2 shows the expression of recombinant CCMV fusion peptides engineered to express PA1, PA2, PA3, and PA4 peptide inserts in the soluble fraction.

### Example 3

### VLP Reassembly

3.A. VLP Reassembly Without RNA:

To assemble the virus-like particles, 50 ml culture of Pseudomonas fluorescens host cells expressing recombinant capsid fusion peptides was French-pressed, and the soluble and insoluble fractions were separated by centrifugation. The insoluble inclusion bodies were washed two times. Samples from the soluble and insoluble fractions were taken and stored at −80° C. The insoluble fraction was resuspended in Buffer B (50 mM Tris pH 7.5, 1M NaCl, 1 mM DTT) containing 8 M urea at 4° C. overnight. The 8 M urea solution was then diluted in 0.25M increments with Buffer B down to a final concentration of 2.0 M urea. Polyethylenimine (PEI) was added to final concentration of 0.033%, and the solution was incubated on ice for 10 minutes. The supernatant was dialyze against Buffer B (3 changes of buffer) to completely remove urea overnight. The supernatant was centrifuged at 27,000×g for 30 minutes.

To determine the final yield of recombinant CCMV capsid fusion peptide, the supernatant was analyzed by absorbance at 280 nm, using an extinction coefficient of 1.20 for free capsid protein to quantify the amount of capsid fusion protein in the solution.

10 uM of capsid fusion peptide solution was dialyzed in Buffer B (use 1 mg of capsid fusion peptide per 4 ml) against Empty Assembly Buffer (50 mM Sodium Acetate pH 5.2, 1M NaCl, 1 mM EDTA, 1 mM DTT) for 2 hrs at 4° C. The assembled particles were washed with Empty assembly buffer using Centricon-100 microconcentrators. The sample retentate containing assembled VLPs was measured by absorbance at 280 nm to determine VLP yield. A portion of the sample was loaded on a sucrose gradient to determine VLP assembly, and the remaining portion was concentrated down in Virus Buffer (0.1M Sodium Acetate, pH.5.2), and run on an SDS-PAGE.

3.B VLP Assembly with RNA:

To assemble the virus-like particles, 50 ml culture of Pseudomonas fluorescens host cells expressing recombinant capsid fusion peptides was French-pressed, and the soluble and insoluble fractions were separated by centrifugation. The insoluble inclusion bodies were washed 2 times. Samples from the soluble and insoluble fractions were taken and stored at −80° C. The insoluble fraction was resuspended in Buffer B (50 mM Tris pH 7.5, 1M NaCl, 1 mM DTT) containing 8 M urea at 4° C. overnight. The 8 M urea solution was then diluted in 0.25M increments with Buffer B down to a final concentration of 2.0 M urea. Polyethylenimine (PEI) was added to final concentration of 0.033%, and the solution was incubated on ice for 10 minutes. The supernatant was centrifuged at 27,000×g for 30 minutes. The supernatant was dialyzed against Buffer B (3 changes of buffer) to completely remove urea overnight.

To determine the final yield of recombinant CCMV capsid fusion peptide, the supernatant was analyzed by absorbance at 280 nm, using an extinction coefficient of 1.20 for free capsid protein to quantify the amount of capsid fusion protein in the solution. A capsid fusion peptide to CCMV RNA ratio of 5:1 weight to weight was used for assembly. The source of RNA was in vitro transcribed CCMV RNA1, RNA2, RNA3, or subgenomic RNA4 or any portion thereof. Alternatively, CCMV viral RNA isolated from plants infected with CCMV can or bromo mosaic virus RNA produced in vitro or in vivo can be used. Alternatively, random mRNA isolated from an organism such as plants or Pseudomonas fluorescens can be used. The concentration of capsid fusion peptides was 10 uM (1 mg in 4 ml). 10 uM of capsid fusion peptide and RNA solution was dialyzed against assembly buffer (50 mM Tris-HCl pH 7.2, 50 mM NaCl, 10 mM KCl, 5 mM MgCl₂, 1 mM DTT) for 2 to 12 hours at 4° C. The resultant assembled particles were washed with assembly buffer using Centricon-100 microconcentrators. A portion of sample retentate was taken and measured by absorbance at 280 nm to determine VLP yield. A portion of the retentate was loaded on a sucrose gradient to determine VLP assembly, and the remaining portion was concentrated down in Virus Buffer (0.1M Sodium Acetate, pH.5.2), and run on an SDS-PAGE.

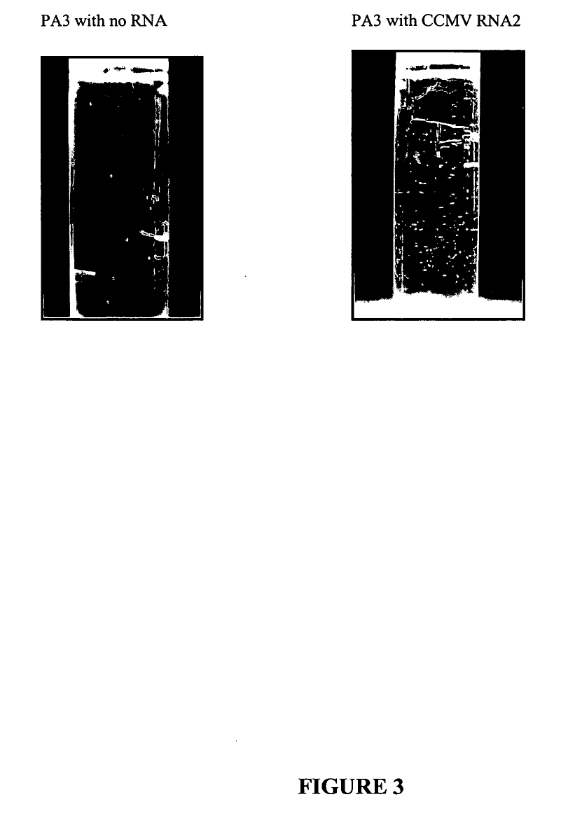

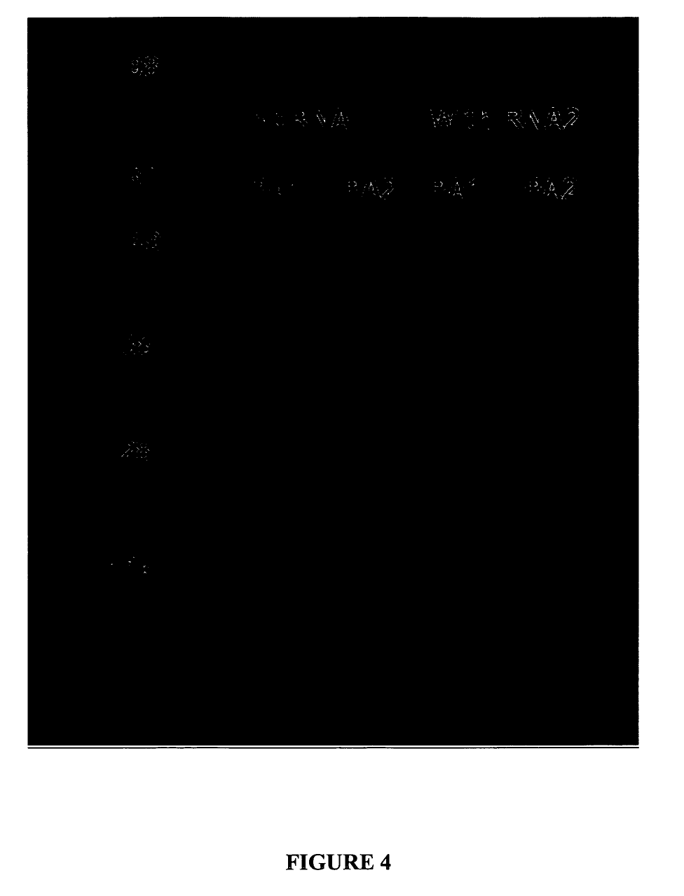

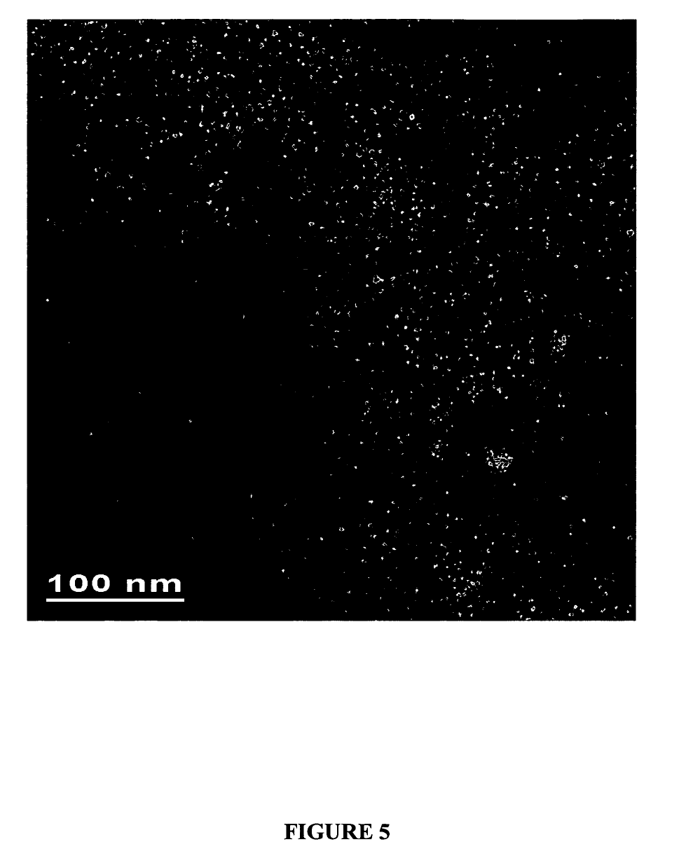

FIG. 3 shows separation of CCMV-PA3 VLPs with and without RNA in a sucrose density gradient. FIG. 4 shows an SDS-PAGE gel of the CCMV-PA1 and CCMV-PA2 VLP bands with and without RNA isolated from the sucrose density gradient. FIG. 5 shows an electron microscopic analysis of VLPs reassembled from CCMV-PA4 capsid fusion peptides in the absence of RNA.

### Example 4

### Re-Assembly of VLPs Containing Multiple Recombinant CCMV-Capsid Fusion Peptides

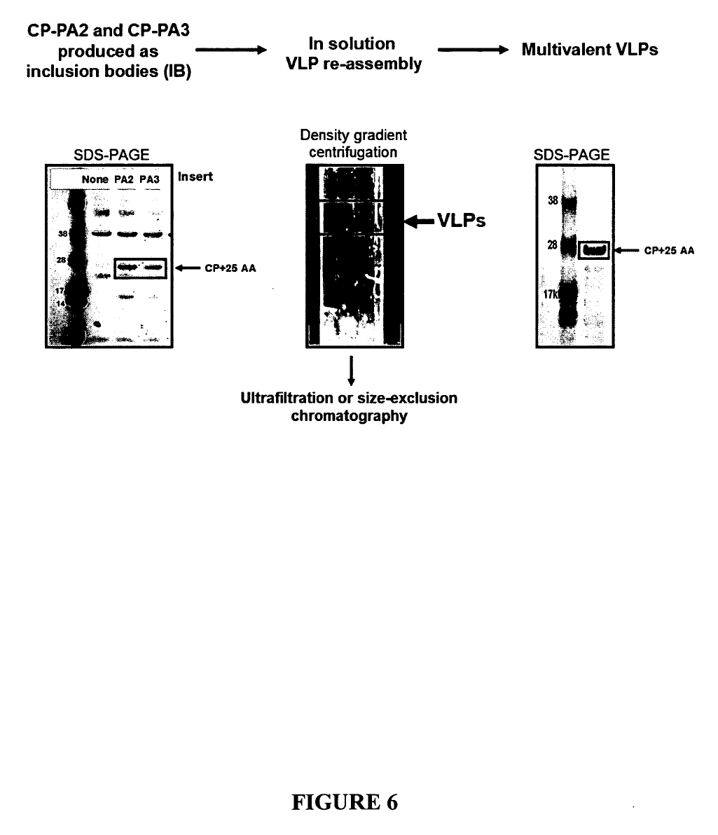

Recombinant CCMV capsid fusion peptides containing antigenic peptides from the same or different pathogens (as described in Example 1) can be produced in Pseudomonas fluorescens as inclusion bodies. The inclusion bodies can be isolated, the various recombinant CCMV-capsid fusion peptides solubilized and refolded as described in Example 3. Various combinations of CCMV-capsid fusion peptides containing antigenic inserts from the same or different pathogenic agents can be mixed in various ratios before assembly. The reassembly reaction can be performed in the presence or absence of RNA as described in Example 3. The resulting multivalent VLPs contain multiple populations of recombinant CCMV-capsid fusion peptides containing different antigenic inserts. The ratios of antigenic peptides can be adjusted by adjusting the amount of each population of recombinant CCMV-capsid fusion peptides containing differing antigenic peptide inserts added to the mixture prior to the assembly reactions. FIG. 6 shows a diagram of expression and reassembly of multivalent VLPs composed of separate recombinant CCMV-capsid fusion peptides containing Protective Antigen-3 (“PA-3”) and PA-4 antigenic peptides.

### Example 5

### Production of CCMV Virus Particles in Plants, Dissasembly of plant Produced CCMV Virus Particles, and Reassembly of Plant CCMV Capsid Protein Into VLPs

Production of CCMV virus particles in plants: Cocktail mixes of CCMV RNA1, RNA2, and RNA3 were used to infect cowpea plants. Cowpea seeds Cowpea California Blackeye #5 seeds (Ferry-Morse Seed Co. KY) were sprouted and transplanted onto 6 inch pots with Miracle-Gro potting mix (Miracle-Gro Lawn Products OH). Cowpea plants were infected at 2-leaf stage (approximately 7 days post germination). A dusting of carborundum powder 400 grit (Fisher Scientific cat.409-21-2) was applied onto one leaf of each plant. RNA cocktail mixes were applied onto the carborundum layer. Leaves were abraded by gentle rubbing with a gloved finger. Infections were established 7-14 days post inoculation. The leaf tissue was harvested and frozen at −80° C. until further processing. Leat tissue was disrupted by blending in virus buffer (0.2M Sodium Acetate pH 5.2; 10 mM EDTA.0). The resulting homogenate was squeezed through three layers of cheese cloth and was then centrifuged for 15 min at 15,000×G at 4° C. The resulting supernatants was removed. To each supernatant, PEG8000 was added to a final concentration of 10% and the solution was incubated on ice for 1 hr or overnight at 4° C. Then, the solution was centrifuged at 15,000×G for 10 min at 4° C. Precipitated pellets were then resuspended in 1/10 initial supernatant volume of virus buffer and the resuspended samples were centrifuged for 10 min at 15,000×G at 4° C. The supernatant was recovered and subjected to the second round of PEG precipitation. PEG8000 was added to final concentration of 15% and stirred at 4° C. for 2 hours. The solution was then centrifuged at 15,000×G for 10 mins and the pellet was resuspended in small volume of virus buffer. The resuspended VLP solution was loaded on to Centricon Plus-20 with 300 K molecular weight cut-off and spinned at 4,000×G for 5 mins.

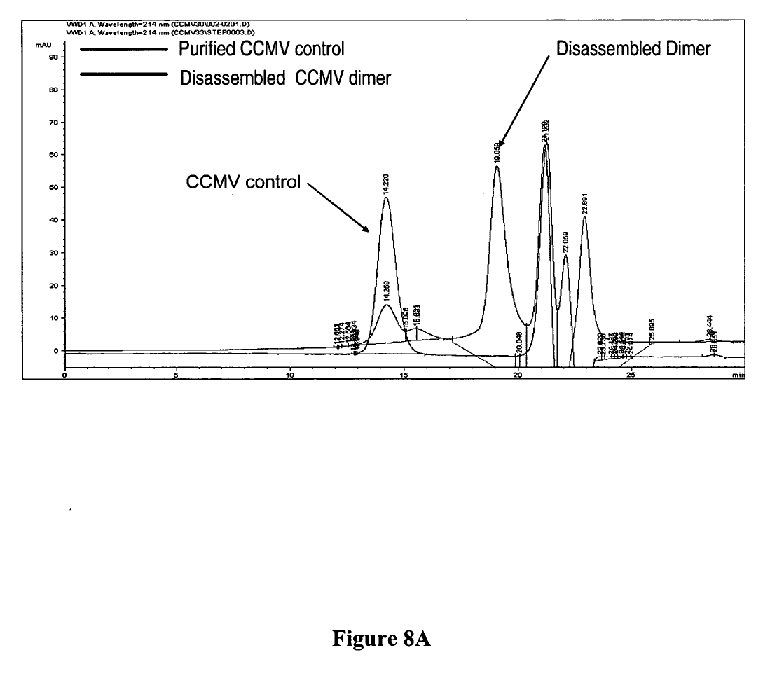

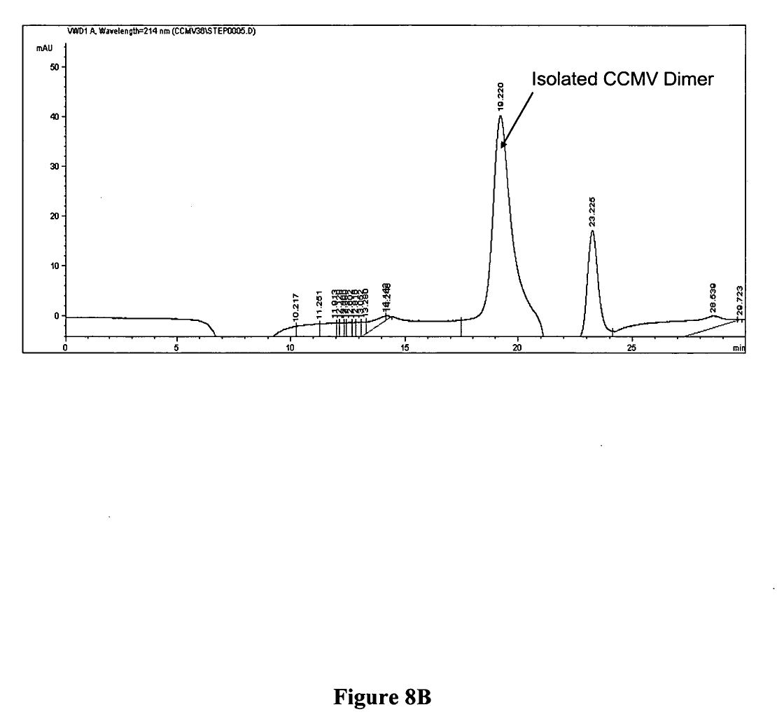

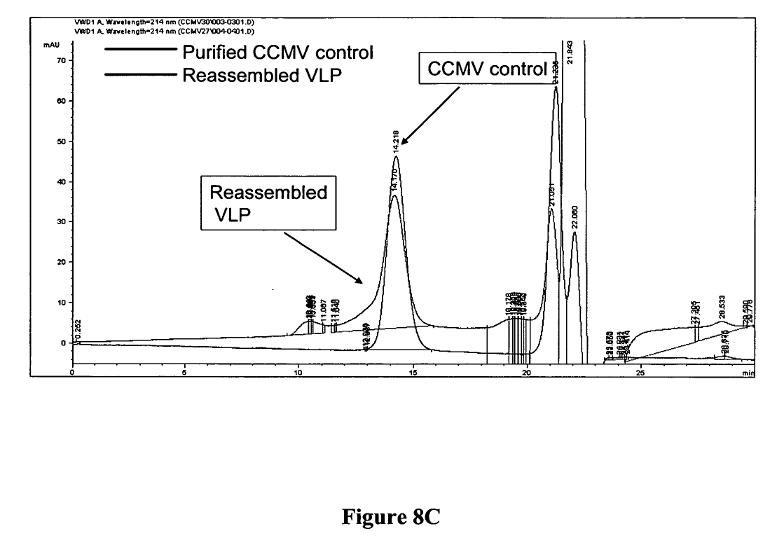

The concentrated VLP sample was then analyzed by SDS-PAGE and western blotting with polyclonal anti-CCMV antibodies. Alternatively, the virus particles were purified on sucrose density gradient. The purified virus particles were analyzed by size exclusion chromatography (SEC)-HPLC (FIG. 8).

Dissasembly of plant produced CCMV virus particles: Purified CCMV was disassembled by dialysis against buffer A (50 mM Tris HCl pH7.5, 500 mM CaCl₂, 1 mM DTT, 0.2 mM PMSF) for 16-29 hours at 4° C. The disassembled virus was centrifuged at 14,000 rpm for 15 min at 4° C. to pellet the viral RNA. The remaining supernatant was dialyzed against buffer B (200 mM Tris HCl pH7.5, 1M NaCl, 1 mM DTT, 0.2 mM PMSF) for 2 hours at 4° C. The CCMV dissasembled into capsid protein dimers that were further purified by FPLC Superose 12 size exclusion chromatography. The diassambled CCMV was analyzed by SEC-HPLC (FIG. 8).

Assembly of CCMV VLPs: CCMV VLPs were assembled by dialyzing the purified dimers overnight against low salt Assembly buffer (100 mM Sodium Acetate pH4.8, 100 mM NaCl, 0.2 mM PMSF) at 4° C. The reassambled CCMV VLPs were analyzed by SEC-HPLC (FIG. 8).

### Example 6

### Disassembly and Reassembly of VLPs Produced in Various Organisms

Previously in vitro or in vivo assembled CCMV particles containing various antigenic inserts from the same of different pathogenic agents can be produced in plants and/or Pseudomonas fluorescens individually. The assembled VLP particles can be isolated and disassembled in vitro. The resultant CCMV-capsid fusion peptides containing the antigenic peptides from the same of different pathogenic agents can be mixed in a predetermined ratio, and subsequently reassembled in the presence or absence of RNA as described in Example 3. The resulting multivalent VLPs are composed of separate recombinant CCMV-capsid fusion peptides and contain multiple inserts. The ratios of antigenic peptides can be adjusted by adjusting the amount of each population of recombinant CCMV-capsid fusion peptides containing differing antigenic peptide inserts added to the mixture prior to the assembly reactions. Wild type capsid protein can be also added to the re-assembly mixture prior to the assembly.

### Example 7

### VLP Re-Assembly in the Presence of CpG

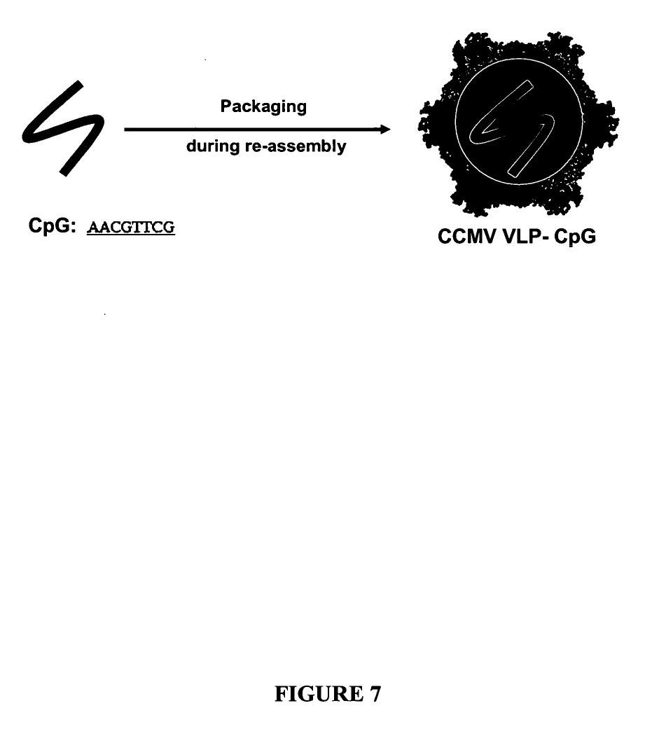

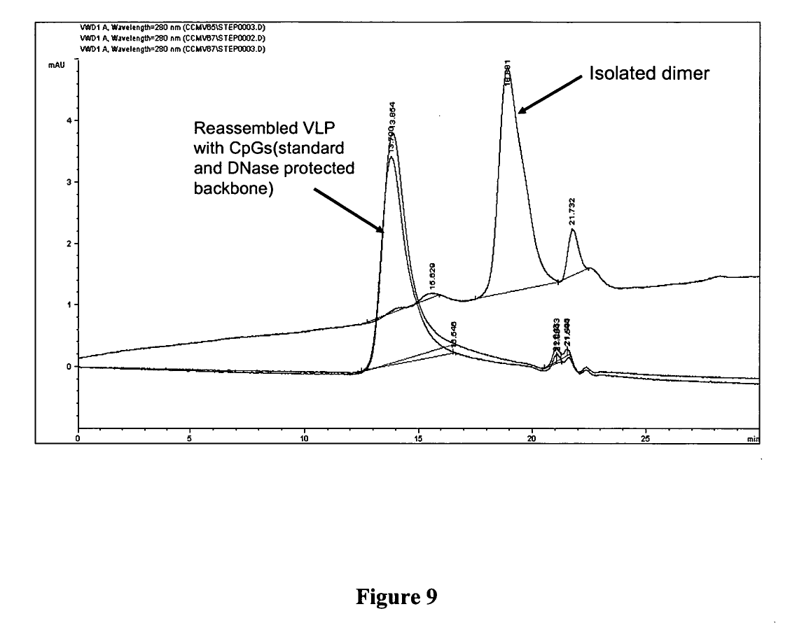

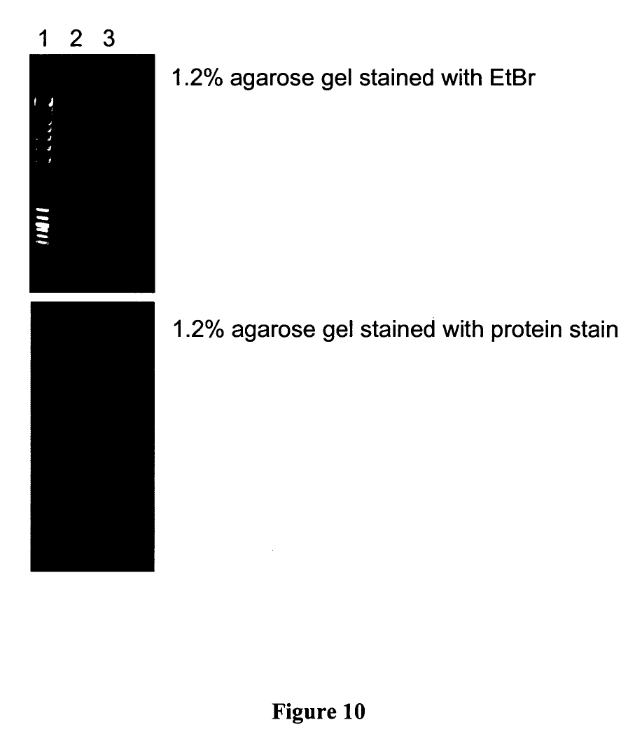

The assembly reaction can be performed in the presence of CpG as shown in FIG. 7. The resulting multivalent VLPs are composed of separate recombinant CCMV-capsid fusion peptides and contain multiple inserts, and further encapsulate CpG inside of the particles. CpGs act as mucosal adjuvant and can induce Th1 immune responses against co-administered antigens. The advantages of encapsulating CpG sequences with VLPs may include lower dosing requirements, a reduction in the side effects associated with CpG co-administration, and increased stability of the CpG and VLP. FIG. 7 shows packaging of CpGs into VLPs during assembly reactions. The plant produced CCMV was diassembled as described in Example 5. The dissasembled CCMV dimers in buffer B (0.5 mg/ml) were mixed with CpG oligonucleotides (120 nmol/ml). Both standard oligonucleotides and oligonucleotides with a DNase-protected backbone were used (Integrated DNA Technologies, Coralville, Iowa). The CpG oligonucleotide sequence was 5′ TCC ATG ACG TTC CTG ACG TT 3′ (SEQ ID NO:23). The solution was dialyzed against assembly buffer (50 mM Tris-HCl pH 7.2, 50 mM NaCl, 10 mM KCl, 5 mM MgCl₂, 1 mM DTT) for 2 to 12 hours at 4° C. as described in Example 3B. The resultant assembled particles were washed with assembly buffer using Centricon-100 microconcentrators and buffer exchanged into Virus Buffer (0.1M Sodium Acetate, pH.5.2). The samples were run on SEC-HPLC (FIG. 9). The results indicated that the dimers assembled into VLPs both in the presence of standard oligonucleotides and in the presence of oligonucleotides with a DNase-protected backbone. The samples were further analyzed on 0.8-1.2% agarose gel. The agarose gel was stained with EtBr to detect the presence of CpG oligonucleotides and subsequently by protein stain to detect the presence of CCMV CP (FIG. 10). The results confirmed that reassembled VLPs encapsulated CpGs inside the particles. Lane 1 is the molecular weight marker, lane 2 shows CCMV VLP sample with encapsulated standard CpGs, and lane 3 shows CCMV VLP sample with encapsulated CpGs containing the DNase-protected backbone.

We claim:

1. A method for producing a multivalent virus like particle comprising:
a) mixing, in vitro:
i) at least one first viral capsid fusion peptide comprising at least one antigenic peptide insert; and,
ii) at least one second viral capsid fusion peptide comprising at least one antigenic peptide insert, wherein at least one second viral capsid fusion peptide comprises at least one antigenic peptide insert that is not present in the first viral capsid fusion peptide; and,
b) assembling the at least one first viral capsid fusion peptide and the at least one second viral capsid fusion peptide to form at least one multivalent virus like particle,
wherein the multivalent virus like particle lacks full length infectious viral nucleic acids.

2. The method according to claim 1, wherein assembling the at least one first viral capsid fusion peptide and the at least one second viral capsid fusion peptide occurs after a purification step.

3. The method according to claim 1, wherein the viral capsid of the first viral capsid fusion peptide and the second viral capsid fusion peptide are derived from the same viral taxa member or from a different viral taxa member.

4. The method of claim 1, wherein the viral capsid of the first and/or second viral capsid fusion peptides are derived from the amino acid sequence of an icosahedral virus.

5. The method of claim 4, wherein the icosahedral virus is cowpea chlorotic mottle virus.

6. The method of claim 1, wherein the antigenic peptide insert of the first and second viral capsid fusion peptide comprise an antigenic peptide insert derived from a pathogenic agent.

7. The method of claim 6, wherein the antigenic peptide inserts of the first capsid fusion peptide and the second capsid fusion peptide are derived from same or from different pathogenic agents.

8. The method of claim 1, wherein the virus like particle lacks viral nucleic acids.

9. The method of claim 1, wherein the first and/or second viral capsid fusion peptide is derived from a virus or virus like particle produced previously in vivo.

10. A method for producing a multivalent virus like particle comprising:
a) mixing, in vitro:
i) at least one first viral capsid fusion peptide comprising at least one antigenic peptide insert,
ii) at least one second viral capsid fusion peptide comprising at least one antigenic peptide insert, wherein the at least one second viral capsid fusion peptide comprises at least one antigenic peptide insert that is not present in the first viral capsid fusion peptide; and
iii) at least one immunostimulatory nucleic acid, wherein the immunostimulatory nucleic acid sequence is a CpG oligonucleotide sequence; and
b) assembling the at least one first viral capsid fusion peptide, the at least one second viral capsid fusion peptide, and the immunostimulatory nucleic acid to form at least one multivalent virus like particle,
wherein the multivalent virus like particle lacks full length infectious viral nucleic acids.

11. The method of claim 10, wherein the CpG oligonucleotide sequence is AACGTTCG (SEQ ID NO:24).

12. The method of claim 10, wherein the viral capsid of the first viral capsid fusion peptide and the second viral capsid fusion peptide are derived from the same viral taxa member or a different taxa member.

13. The method of claim 10, wherein the viral capsid of the first and/or second viral capsid fusion peptides is derived from the amino acid sequence of an icosahedral virus.

14. The method of claim 13, wherein the icosahedral virus is cowpea chlorotic mottle virus.

15. The method of claim 10, wherein the antigenic peptide insert of the first and second viral capsid fusion peptide comprise an antigenic peptide insert derived from a pathogenic agent.

16. The method of claim 15, wherein the first capsid fusion peptide and the second capsid fusion peptide comprise antigenic peptide inserts derived from the same or different pathogenic agents.

17. A method for producing a multivalent virus like particle comprising:
a) providing:
i) at least one first virus like particle comprising at least one first capsid fusion peptide comprising at least one antigenic peptide insert; and,
ii) at least one second virus like particle comprising at least one second capsid fusion peptide comprising at least one antigenic peptide insert;
b) disassembling:
i) the first virus like particle to provide at least one isolated first viral capsid fusion peptide comprising at least one antigenic peptide insert; and,
ii) the second virus like particle to provide at least one isolated second capsid fusion peptide comprising at least one antigenic peptide insert, wherein the at least one second viral capsid fusion peptide comprises at least one antigenic peptide insert that is not present in the first viral capsid fusion peptide; and
c) mixing, in vitro:
i) the at least one first viral capsid fusion peptide; and
ii) the at least one second viral capsid fusion peptide; and
d) assembling the at least one first viral capsid fusion peptide and the at least one second viral capsid fusion peptide to form at least one multivalent virus like particle,
wherein the multivalent virus like particle lacks full length infectious viral nucleic acids.

18. The method of claim 17, wherein the viral capsid of the first viral capsid fusion peptide and the second viral capsid fusion peptide are derived from the same or different viral taxa members.

19. The method of claim 17, wherein the viral capsid of the first viral and/or second capsid fusion peptide are derived from the amino acid sequence of an icosahedral virus.

20. The method of claim 19, wherein the icosahedral virus is a cowpea chlorotic mottle virus.

21. The method of claim 17, wherein the antigenic peptide insert of the first and second viral capsid fusion peptide comprises an antigenic peptide insert derived from a pathogenic agent.

22. The method of claim 17, wherein the first capsid fusion peptide and the second capsid fusion peptide comprise different antigenic peptide inserts derived from the same or different pathogenic agents.

23. The method of claim 17, wherein the first and/or second capsid fusion peptide is derived from expression in Pseudomonas fluorescens.

24. The method according to claim 17, further comprising mixing, in vitro, iii) at least one immunostimulatory nucleic acid sequence, wherein the immunostimulatory sequence is a CpG oligonucleotide sequence.

25. The method according to claim 24, wherein the CpG oligonucleotide sequence is AACGTTCG (SEQ ID NO:24).

26. A multivalent virus like particle comprising:
i) at least one first cowpea chlorotic mottle virus capsid fusion peptide comprising at least one antigenic peptide insert; and,
ii) at least one second cowpea chlorotic mottle virus capsid fusion peptide comprising at least one antigenic peptide insert, wherein at least one second viral capsid fusion peptide comprises at least one antigenic peptide insert that is not present in the first viral capsid fusion peptide; and
wherein the multivalent virus like particle lacks full length infectious viral nucleic acids.

27. The multivalent virus like particle of claim 26, wherein the first capsid fusion peptide and the second capsid fusion peptide comprise an antigenic peptide insert where the antigenic peptide inserts are derived from the same or different pathogenic agents.

28. The multivalent virus like particle of claim 26, wherein the resultant multivalent virus like particle lacks viral nucleic acids.

29. The multivalent virus like particle of claim 26, wherein the virus like particle comprises a CpG oligonucleotide sequence.

30. The multivalent virus like particle of claim 26, wherein the CpG oligonucleotide sequence comprises AACGTTCG (SEQ ID NO:24).

31. A method of increasing the solubility of a multivalent virus like particle comprising mixing, in vitro, at least one first viral capsid fusion peptide comprising at least one antigenic peptide insert, and at least one second viral capsid fusion peptide comprising at least one antigenic peptide insert, wherein at least one second viral capsid fusion peptide comprises at least one antigenic peptide insert that is not present in the first viral capsid fusion peptide, and assembling the at least one first viral capsid fusion peptide and the at least one second viral capsid fusion peptide to form at least one multivalent virus like particle, wherein the resultant multivalent virus like particle lacks full length infectious viral nucleic acids.

32. A vaccine comprising the multivalent virus like particle of claim 1.
